(19) United States
(12) Patent Application Publication
(10) Pub. No.: US 2024/0398836 A1
(43) Pub. Date: Dec. 5, 2024

(54) EPOXY STEROIDS

(71) Applicant: Helios Klinken Schwerin GmbH, Schwerin (DE)

(72) Inventors: Aram PROKOP, Schwerin (DE); Hans-Günther SCHMALZ, Brühl (DE); Ömer TASPINAR, Köln (DE); Jennifer WITT, Quickborn (DE); Tobias WILCZEK, Bergisch Gladbach (DE)

(21) Appl. No.: 18/722,018

(22) PCT Filed: Dec. 22, 2022
(86) PCT No.: PCT/EP2022/087575

§ 371 Date: Jun. 20, 2024

(30) Foreign Application Priority Data
Dec. 22, 2021 (EP) 21217028.6

(51) Int. Cl. A61K 31/58; A61K 31/475; A61K 31/7068; A61P 35/00; C07J 71/00

(52) U.S. Cl. CPC A61K 31/58; A61K 31/475; A61K 31/7068; A61P 35/00; C07J 71/0005

(57) ABSTRACT

A drug on the basis of 7,19-epoxy steroids, comprising a compound of the general formula (I)

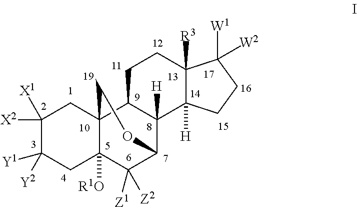

or a physiologically acceptable salt thereof,
wherein R¹=H; methyl, aryl-CH₂—, R^{x}—C(═O)— or R^{x}—O—C(═O)—, or R¹O is dispensed with if there is a double bond between 4-5,
W¹, W², independently of each other, represent H, OH, CHR²OH, CHR²R^{x}, CR²R^{x}OH or W¹ and W² together represent O,
   R²=saturated or unsaturated, optionally branched alkyl groups with 4 to 9 C atoms, wherein these alkyl groups can also have a cyclopropane or cyclobutane substructure;
R³=methyl or ethyl,
X¹, X² independently of each other represent R^{x} or OR⁵, or together represent O, NOR⁴;
Y¹, Y² independently of each other represent R^{x} or OR⁵, or together represent O, NOR⁴;
or X¹ and Y¹ together form a double bond or an epoxy group;
Z¹, Z² independently of each other represent R^{x} or OR⁵, or together represent O, NOR⁴, CH₂;
   R⁴ independently of each other represent H, alkyl or aryl; and
   R⁵ independently of each other represent H; alkyl, aryl-CH₂—, R^{x}—C(═O)— or R^{x}—O—C(═O)—, SO₃H, SO₃CH₃, PO(OH)₂ or glycosyl;
with each R^{x} independently of each other representing H, saturated or unsaturated, optionally branched alkyl groups, saturated or unsaturated, optionally branched substituted alkyl, aryl, heteroaryl, PEG, SO₃H, PO(OH)₂, or glycosyl;
and wherein up to 3 hydrogens can be substituted by fluorine.

The present invention relates to new medicaments based on steroids.

The formation of malignant tumors and leukemias and other, benign, hyperproliferative diseases, such as psoriasis or keloid, results from a disturbed balance between new tissue formation and the regulated dying of cells from the coherent tissue.

In clinical practice, by using cyrotoxic treatment methods, such as chemotherapy, radiotherapy and hyperthermy, it is tried to restore this disturbed balance, and to simultaneously kill the excess tumor cells. It is generally recognized that most chemotherapeutics currently employed in clinical practice display their activity by inducing apoptosis (programmed cell death) (Hannun, 1997). However, part of the patients affected by malignant tumors develop resistencies against chemotherapy or radiation early on, or are primarily refractory to therapy (Hickman, 1996). It is further known that the primary tumor and the metastases often respond quite differently to cytotoxic therapies.

Because of more recent studies, it is known that the causes of resistances often reside in different disturbances of the apoptotic signal cascade (Raisova et al., 2000).

In particular, the therapies of recurrences of leukemias, lymphomas and solid tumors in childhood still do not show any satisfactory survival rates. Thus, for example, despite of aggressive therapies, about 45% of the affected children die in a recurrent acute lymphatic leukemia (ALL). It is similar with other, in part hard to treat, tumor diseases, such as brain tumors, lymphomas, sarcomas, such as soft-tissue sarcomas, Ewing sarcoma, osteosarcoma, germ cell tumors, kidney or liver tumors and carcinomas, such as thyroid carcinoma, or colon carcinomas and mamma carcinomas, which are very frequent in adult age.

Further, there is no suitable chemotherapy for difficult and rare tumors, such as desmoblastic round cell tumor, or malignant peripheral nerve sheath tumor, in the case of metastasis, because these malignant cells have resistance to cytostatic drugs.

Further, psoriasis is one of the most frequent skin diseases (two to four percent of humans suffer from it), whose therapy is still worthy of improvement.

Although a multitude of medicaments displaying their activity through apoptosis are known, there is still a need for novel medicaments that can overcome the resistance of malignant cells against conventional cytostatic drugs.

Further, there is a large need for novel cytostatic agents that can intrude into the central nervous system, because they have a high distribution volume. This is of great importance especially for treating different brain tumors (grade 1 to grade 4), since many conventional cytostatic drugs cannot cross the blood-brain barrier in sufficient amounts, so that the necessary concentration of active ingredient in the tumor tissue cannot be reached in a chemotherapy. Conventional steroids can cross the blood-brain barrier easily, but do not show any cytostatic effect in brain tumors, other solid tumors, or in steroid-resistant leukemias and lymphomas, or not sufficiently so.

It has been the object of the present invention to provide medicaments that overcome the above drawbacks of the prior art.

The object is achieved by medicaments based on 7,19-epoxy-steroids comprising a compound of general formula T,

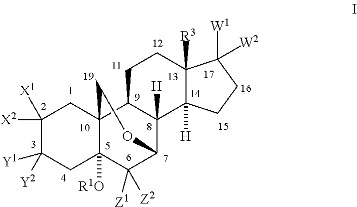

or a physiologically tolerable salt thereof, wherein in the formula:
R¹=H; methyl, aryl-CH₂—, R^{x}—C(═O)—, or R^{x}—O—C(═O)—; or R¹O is omitted if a double bond exists between 4-5;
W¹, W² are independently H, OH, CHR²OH, CHR²R^{X}, CR²R^{x}OH, or W¹ and W² are together O,
   R²=saturated or unsaturated, optionally branched, alkyl radicals with 4 to 9 carbon atoms, wherein such alkyl radicals may also have a cyclopropane or cyclobutane substructure;
R³=methyl or ethyl,
X¹, X² are independently R^{x} or OR⁵, or together form O, NOR⁴;
Y¹, Y² are independently R^{x} or OR⁵, or together form O, NOR⁴;
or X¹ and Y¹ together form a double bond or epoxy group;
Z¹, Z² are independently R^{x} or OR⁵, or together form O, NOR⁴, CH₂;
   R⁴ are independently H, alkyl or aryl; and
   R⁵ are independently H; alkyl, aryl-CH₂—, R^{x}—C(═O)— or R^{x}—O—C(═O)—, SO₃H, SO₃CH₃, PO(OH)₂ or glycosyl;
wherein each R^{x} is independently H, saturated or unsaturated, optionally branched, alkyl, saturated or unsaturated, optionally branched, substituted alkyl, aryl, heteroaryl, PEG, SO₃H, PO(OH)₂, or glycosyl;
and wherein up to 3 hydrogens may be substituted by fluorine.

In a preferred embodiment, the object is achieved by medicaments based on 7,19-epoxy-steroids comprising a compound of general formula A,

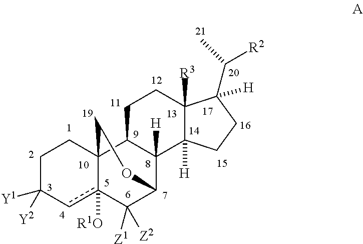

or a physiologically tolerable salt thereof, wherein in the formula:
R¹=H; methyl, aryl-CH₂—, R^{x}—C(═O)—, or R^{x}—O—C(═O)—; or wherein R¹O is omitted if a double bond exists between 4-5;
R²=saturated or unsaturated, optionally branched, alkyl radicals with 4 to 9 carbon atoms, wherein such alkyl radicals may also have a cyclopropane or cyclobutane substructure;
R³=methyl or ethyl;
Y¹, Y²=(H/OR⁵), or together form O, NOR⁴;
Z¹, Z²=(H/OR⁵), or together form O, NOR⁴;
R⁴ are independently H, alkyl or aryl; and
R⁵ are independently H; alkyl, aryl-CH₂—, R^{x}—C(═O)— or R^{x}—O—C(═O);
wherein each R^{x} is independently H, saturated or unsaturated, optionally branched, alkyl, saturated or unsaturated, optionally branched, substituted alkyl, aryl, heteroaryl, PEG, SO₃H, PO(OH)₂, or glycosyl;
and wherein up to 3 hydrogens may be substituted by fluorine.
Formula A also includes the basic structures A1 and A2:

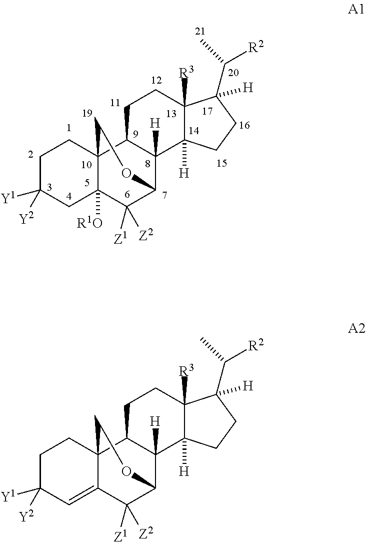

The compounds according to the invention are constructed on the basic structure of steroids that are well researched both synthetically and medicinally/pharmacologically.

In preferred embodiments, R^{x} has 1 to 10 carbon atoms in formula I or A.

In preferred embodiments, R^{x} is a substituted alkyl in formula I or A. Preferred substituents include —OR′ or C(═O)OR′, and R′ is H or alkyl or a protective group.

Preferred protective groups include carbonate, such as tBOC (tert-butyloxycarbonyl), and silylether, such as TBDMS (tert-butyldimethylsilyl), esters or acetals, such as MOM (methoxymethyl). Further protective groups are known to those skilled in the art from Greene's Protective Groups in Organic Synthesis, fourth edition, which is completely included herein by reference.

The compounds according to the invention differ from other steroids, in particular, by the 7,19-epoxy link.

These substances induce apoptotic cell death not only in leukemia and lymphoma cells, but also in solid tumor cells, and in leukemia and lymphoma cells that are resistant to steroids, or polychemotherapy-resistant. Because of their lipophilic nature, these compounds of general formula I or A may be employed against tumor diseases of bone marrow, but also against tumors of different origins, such as epithelial tumors, sarcomas, or malignant diseases of the skin, etc.

Crossing the blood-brain barrier seems to be possible with the developed substances because of their lipophilic nature, and therefore, these substances may also be employed for malignant brain tumors (grade 1 to grade 4), such as gliomas, astrocytomas, medulloblastomas, or glioblastomas.

Because of the large distribution volume of the steroids, presumably, tumor metastases in the regions of the human body that are difficult to access for cytostatic drugs, such as the pleural cavity, peritoneum, mediastinum, etc., may also be addressed in a cytostatic therapy with these novel active substances.

According to the invention, studies of the apoptotic signal cascade enabled the development of novel steroids, which, in contrast to the conventional glucocorticoids, induce apoptosis in malignant cells on all three known pathways of the apoptotic signal cascade (receptor-mediated, mitochondrial, and mediated by the endoplasmic reticulum), and thus can destroy leukemia, lymphoma and solid tumor cells that are resistant to glucocorticoids, or polycytostatic-resistant. This effect is selective for malignant cells.

In a preferred embodiment of the invention, R¹ is H, Z¹, Z² are O, R³ is methyl.

In another preferred embodiment of the invention, Y¹, Y² are (H/OH), or Y¹, Y² are (H/OR⁵), wherein R⁵ is R^{x}—C(═O)— or R^{x}—O—C(═O)—, and R^{x} is H, alkyl, substituted alkyl, aryl, heteroaryl, wherein each of the individual positions may also be fluorinated.

In a preferred embodiment of the invention, R^{x} is H, alkyl, and R² is 4-methylpentyl.

Preferred substituents for R¹ include H or methyl.

Preferably, each R⁴, R⁵ and R^{x} independently have up to 10 carbon atoms.

In preferred embodiments, R^{x} is alkyl or substituted alkyl.

Unless defined otherwise, substituents preferably have 1 to 10 carbon atoms. The invention also relates to a process for the production of compounds of formula I, comprising step a, followed by conversion of the functional groups:

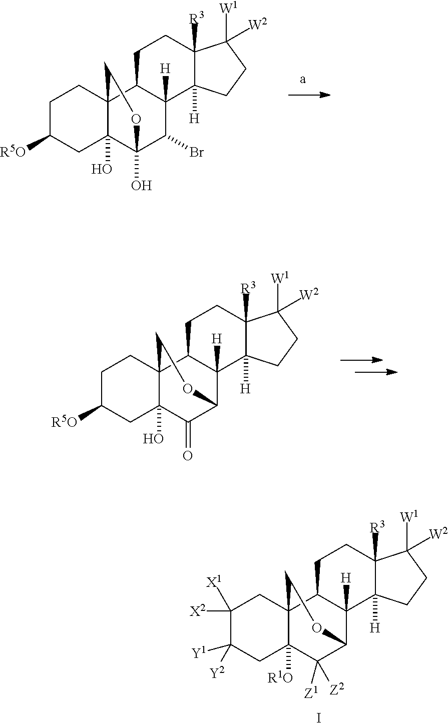

wherein step a is performed under basic conditions.

The invention preferably relates to a process for the production of compounds of formula A, comprising step a, followed by conversion of the functional groups:

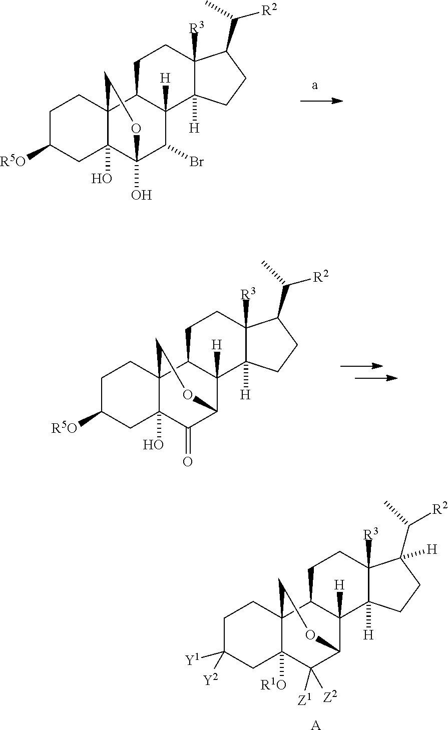

wherein step a is performed under basic conditions, preferably by heating the substrate in the presence of Li₂CO₃ and LiBr in N,N-dimethylformamide at temperatures of 80 to 120° C.

The invention also relates to the use of the medicaments according to the invention in the therapy of malignant diseases, benign or semimalignant skin diseases.

Typical malignant diseases include diseases relating to the bone marrow or other hematopoietic organs, or are solid tumors or epithelial tumors, wherein the solid tumors are, in particular, brain tumors, such as glioblastoma and medulloblastoma.

Typical benign or semimalignant diseases include skin diseases, such as psoriasis vulgaris, keloids, or basaliomas.

The medicaments according to the invention are also suitable for use in the therapy of inflammatory and chronic inflammatory diseases, for application to immunosuppressive therapy, to antiviral, antibacterial, antimycotic, antiprotozoan, or antihelminthic therapy.

The compounds are preferably administered intravenously in a concentration range of from 0.1 to 100 μg/ml, based on the patient's blood volume.

In a topical application, 0.1 to 5% by weight, based on the finished preparation, is rubbed into the diseased skin.

Oral intake is also possible for the compounds.

As shown in the following Examples, the compounds according to the invention are characterized by the following properties:

Differences in effects as compared to conventional glucocorticoids

| | Compounds | |
|---|---|---|
| | Formula I/A | Glucocorticoids |
| Induction of necrosis (LDH) | no | no |
| Inhibition of proliferation | +++ | + |
| Overcoming of cytostatics | 8/8 | 0/8 |
| resistance | | |
| Involvement of intrinsic | yes | yes |
| apoptotic signal pathway | | |
| Involvement of extrinsic | yes | yes? |
| apoptotic signal pathway | | |
| ROS induction | no | yes |
| ER stress induction | yes | yes (protective) |
| Autophagy | yes (protective) | yes (protective) |

In contrast to conventional steroids, the novel group of 7,19-epoxy steroids shows a cytostatic effect in solid tumor cells, in different brain tumor cells, and in steroid-resistant leukemia and lymphoma cells that bear co-resistances to a wide variety of conventional cytostatic drugs used in therapy. Also, an effect in cytostatic drugs-resistant solid tumor cells can be demonstrated.

The compounds are also effective in pancreatic carcinoma cells.

This novel steroid class specifically induces apoptosis in malignant cells, while primary human leukocytes are not damaged.

The novel steroids show a synergistic effect in vitro with conventional cytostatic drugs used in the therapy of malignant diseases.

The overcoming of cytostatic drugs resistance in combination with the large distribution volume of the active ingredients in the human body caused by its steroid structure are the particular new properties of this new class of active substances, which are promising in terms of a good cytostatic effect in malignant diseases that have been hard or unable to treat.

### FIGURES

FIG. 1 shows the inhibition of proliferation in Nalm-6 cells.

FIG. 2 shows the overcoming of steroid and cytostatic resistances in NaKu cells.

FIG. 3 shows the overcoming of polycytostatic resistance in BiBo cells.

FIG. 4 shows the overcoming of polycytostatic resistance in 7-CCA cells.

FIG. 5 shows synergistic effects with cytarabin and vincristin on Nalm-6 cells.

FIG. 6 shows the exclusion of unspecific necrosis in Nalm-6 cells.

FIG. 7 shows the selectivity for leukemia cells (Nalm-6 cells) and lymphoma cells (BJAB cells) as compared to human leukocytes.

FIG. 8 shows the effect in cytostatics-resistant solid tumors.

FIG. 9 shows the effect in brain tumor cells.

FIG. 10 shows the effect in primary human glioblastoma cells.

FIG. 11 shows the effect in primary human pancreatic carcinoma cells.

FIG. 12 shows the distribution of the substances according to the invention in serum.

FIG. 13 shows the distribution of the substances according to the invention in the brain.

FIG. 14 shows the distribution of the substances according to the invention in the liver.

### EXAMPLES

The invention is further illustrated by the following Examples.

### Example 1: Synthesis of 3β,5α-dihydroxy-7β,19-epoxy-cholestan-6-one (9, WIL-071)

The synthetic sequence for preparing the novel steroids according to the invention is illustrated for the substance WIL-071 as an example. At first, the C-19 methyl group of cholesteryl acetate (1) is hydroxylated according to a method basically known from the literature, before the 19-hydroxycholesteryl acatate (4) is oxidatively functionalized in the B cycle, and the 7,19-epoxy bridge is closed.

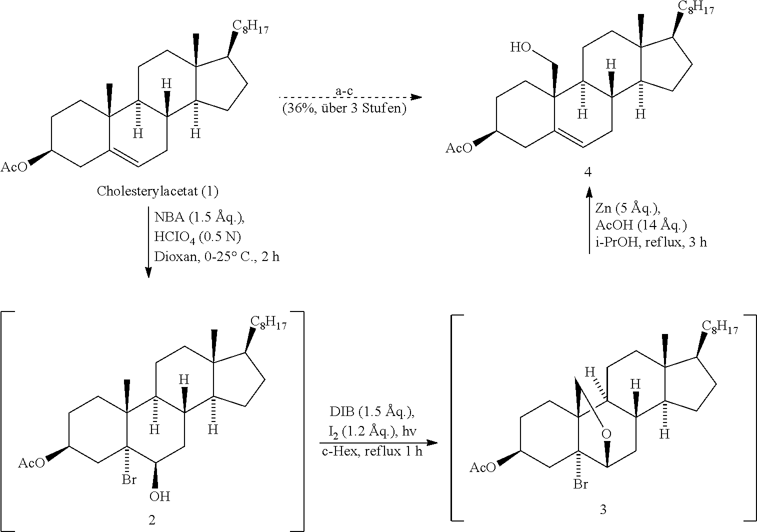

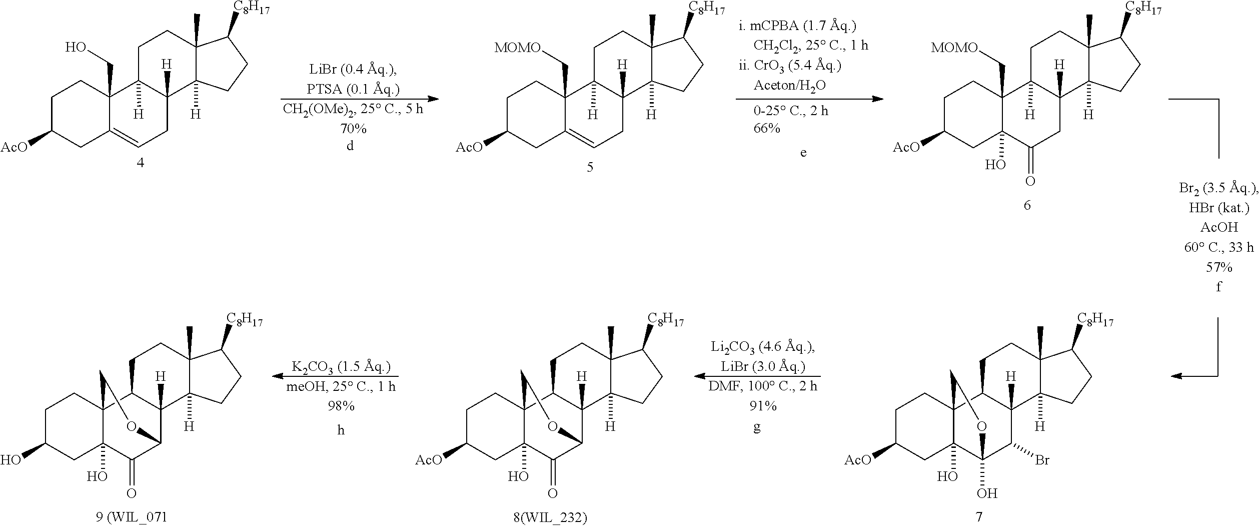

### Steps a-c: Synthesis of 3β-acetoxy-cholest-Δ⁵-en-19β-ol (4)

200 g (0.23 mol, 1.0 eq.) of cholesteryl acetate (1) in 1.6 liters of dioxan was admixed with 96 g (0.67 mol, 1.5 eq.) of N-bromoacetamide (NBA) and 320 ml of HClO₄ (0.5 N). After 1 h at 0° C. in the dark and 1 h at room temperature, the reaction was stopped by adding a saturated Na₂SO₃ solution (until the reaction mixture changes its color) and water. After extraction with MTBE, the combined organic phases were washed with saturated NaCl solution and dried over MgSO₄. Subsequently, the solvent was removed under reduced pressure. The raw product (2, beige solid), with considerable amounts (>40%) of regio-isomeric bromohydrin, was employed in the following step without further purification. For a detailed analysis, a small amount of the bromohydrin 2 was purified by column chromatography.

To a solution of (about 25 g) bromohydrin 2 (+regioisomer) in 1.5 L of cyclohexane were added 27.6 g (85.8 mmol, 1.5 eq.) of diacetoxyiodobenzene (DIB) and 8.76 g (68.6 mmol, 1.2 eq.) of iodine. The reaction mixture was heated under reflux for 1 h under irradiation by a 150 Watt mercury vapor lamp. The purple reaction mixture was cooled to room temperature, and quenched by adding a saturated Na₂SO₃ solution and water (until the color changes). After extraction with EtOAc, the combined organic phases were washed with saturated NaCl solution and dried over MgSO₄. Subsequently, the solvent was removed under reduced pressure, and the raw product (3, brown viscous oil) could be employed in the next step without further purification. This procedure was performed for a total of seven times. A small amount of 3 was purified by column chromatography for analytical purposes.

To a solution of compound 3 (about 50 g) in 1.6 L of i-PrOH were added 37.8 g (0.58 mol, 5.0 eq.) of zinc powder and 94 mL (98 g, 1.64 mol, 14.0 eq.) of acetic acid. The reaction mixture was heated under reflux for 3 h, then cooled to room temperature, and filtered through Celite©. The clear yellow solution was concentrated to a volume of 100 mL, and extracted with MTBE after the addition of water. The combined organic phases were washed with saturated NaCl solution and dried over MgSO₄. The solvent was removed under reduced pressure. This procedure was performed for a total of four times. The product 4 was obtained after purification by column chromatography on silica gel (c-hex/EtOAc, 10:1) in the form of a beige solid (74 g, 0.17 mol, 36% over three stages).

Melting point: 112° C.-114° C.

¹H NMR (500 MHz, CDCl₃): δ [ppm]=5.76 (d, J=5.0 Hz, 1H), 4.66-4.60 (m, 1H), 3.82 (d, J=11.2 Hz, 1H), 3.61 (dd, J=11.4, 9.1 Hz, 1H), 2.41 (ddd, J=13.0, 4.9 Hz, 2.1 Hz, 1H), 2.28-2.23 (m, 1H), 2.02 (s, 3H), 1.95 (dt, J=13.8, 3.4 Hz, 1H), 1.90-0.93 (m, 26H), 0.90 (d, J=6.5 Hz, 3H), 0.85 (dd, J=6.6, 2.2 Hz, 3H), 0.72 (s, 3H).

¹³C NMR (125 MHz, CDCl₃): δ [ppm]=170.6, 134.7, 128.4, 73.6, 62.9, 57.7, 56.2, 50.4, 42.7, 41.7, 40.1, 39.6, 38.4, 36.3, 35.9, 33.5, 33.2, 31.4, 28.4, 28.2, 28.2, 24.2, 24.0, 23.0, 22.7, 21.9, 21.5, 18.8, 12.4.

FT-IR (ATR): ν [cm⁻¹]=3507 (bw), 2944 (s), 2934 (s), 2870 (m), 1734 (m), 1713 (s), 1469 (m), 1444 (m), 1381 (m), 1370 (m), 1254 (s), 1245 (vs), 1029 (vs), 977 (m), 961 (m), 911 (w), 886 (w), 824 (w), 807 (w), 742 (w), 670 (w), 610 (w).

HR-MS: (ESI, 70 eV)=m/z calculated for C₂₉H₄₈O₃Na⁺ [M+Na]⁺ 467.3496 u, found: 467.3493 u.

### Step d: Synthesis of 3β-acetoxy-19-methoxymethyloxy-cholestan-Δ⁵-ene (5)

42 g (78.7 mmol, 1.0 eq.) of 19-hydroxycholesteryl acetate (4) in 315 mL of 5 CH₂(OMe)₂ was admixed with 1.64 g (18.9 mmol, 0.2 eq.) of lithium bromide and 1.8 g (9.44 mmol, 0.1 eq.) of p-toluenesulfonic acid (as monohydrate), and the reaction mixture was stirred at room temperature for 5 h. After complete conversion was observed (TLC control), the reaction was stopped by adding a saturated NaCl solution, and the aqueous phase was extracted with MTBE. The combined organic phases were washed with a saturated NaHCO₃ solution and an NaCl solution, and dried over MgSO₄. The product 5 could be obtained after purification by column chromatography on silica gel (c-hex/EtOAc, 5:1) in the form of a white solid (26.9 g, 55.0 mol, 70%).

Melting point: 84° C.-85° C.

¹H NMR (500 MHz, CDCl₃): δ [ppm]=5.60 (d, J=5.4 Hz, 1H), 4.67-4.58 (m, 3H), 3.73 (d, J=10.3 Hz, 1H), 3.48 (d, J=10.3 Hz, 1H), 3.37 (s, 3H), 2.40 (ddd, J=13.0, 5.2, 2.2 Hz, 1H), 2.33 (t, J=12.2 Hz, 1H), 2.11 (dt, J=13.8, 3.7 Hz, 1H), 2.03 (s, 3H), 2.02-0.96 (m, 25H), 0.91 (d, J=6.5 Hz, 3H), 0.86 (dd, J=6.6, 2.3 Hz, 6H), 0.70 (s, 3H).

¹³C NMR (125 MHz, CDCl₃): δ [ppm]=170.7, 135.8, 126.2, 97.0, 73.8, 69.1, 57.4, 56.2, 55.6, 50.5, 42.6, 40.5, 40.2, 39.7, 38.5, 36.3, 36.0, 33.4, 32.9, 31.7, 28.4, 28.2, 28.2, 24.3, 24.0, 23.0, 22.7, 22.0, 21.6, 18.8, 12.2.

FT-IR (ATR): ν [cm⁻¹]=3419 (br w), 2932 (m), 2868 (m), 1733 (m), 1498 (w), 1467 (w), 1444 (w), 1366 (m), 1242 (s), 1144 (m), 1112 (m), 1096 (m), 1046 (s), 1033 (s), 986 (m), 962 (m), 943 (m), 915 (m), 881 (w), 845 (w), 824 (w), 814 (w), 727 (w), 703 (w), 679 (w), 630 (w), 607 (m), 580 (w).

### Step e: Synthesis of 3β-acetoxy-5α-hydroxy-19-methoxymethyloxy-cholestan-6-one (6)

32.4 g (66.3 mmol, 1.0 eq.) of compound 5 was dissolved in 400 mL of CH₂Cl₂, and admixed with 25.3 g (113 mmol, 1.7 eq.) of mCPBA (70%). The reaction mixture was stirred at room temperature for 1 h (when a complete conversion was reached, a white precipitate was observed, which is not necessarily required, however), and cooled to 0° C. The white precipitate formed was dissolved by adding 950 mL of acetone, and the clear solution was admixed with a solution of 24 g (239 mmol, 3.6 eq.) of CrO₃ in 80 mL of water. After 10 minutes at 0° C., the reaction mixture was taken to room temperature, and stirred for 1 h. Subsequently, the previously described procedure was repeated with 7.82 g (78.2 mmol, 1.18 eq.) of CrO₃ in 35 mL of water. After 2 h, the reaction mixture was quenched by adding a saturated NaHCO₃ solution, and the aqueous phase was extracted with EtOAc. The combined organic phases were washed with water, a saturated NaHCO₃ solution and a saturated NaCl solution. The clear yellow solution was dried over MgSO₄, and the solvent was removed under reduced pressure. Subsequently, the desired product 6 was obtained after purification by column chromatography on silica gel (c-hex/EtOAc, 8:1) in the form of a white foam (22.3 g, 42.8 mol, 65%).

Melting point: 143° C.-144° C.

¹H NMR (500 MHz, CDCl₃): δ [ppm]=5.11 (m, 1H), 4.50 (s, 2H), 3.64 (d, J=10.6 Hz, 1H), 3.57 (d, J=10.6 Hz, 1H), 3.34 (s, 3H), 2.75 (s, 1H), 2.57 (dd, J=14.6, 11.6 Hz, 1H), 2.20-2.13 (m, 2H), 2.09-2.03 (m, 2H), 2.02 (s, 3H), 1.96-1.82 (m, 3H), 1.76-1.63 (m, 3H, H-1), 1.59-1.43 (m, 5H), 1.39-0.96 (m, 12H), 0.91 (d, J=6.5 Hz, 3H), 0.86 (dd, J=6.6, 2.1 Hz, 6H), 0.68 (s, 3H).

¹³C NMR (125 MHz, CDCl₃): δ [ppm]=209.6, 171.1, 97.1, 77.8, 70.4, 67.1, 57.0, 56.3, 56.1, 45.7, 44.1, 43.2, 41.5, 40.0, 39.6, 37.2, 36.3, 35.9, 32.5, 28.3, 28.1, 26.6, 26.1, 24.0, 22.9, 22.7, 21.9, 21.5, 18.8, 12.3.

FT-IR (ATR): ν [cm⁻¹]=3384 (br w), 2939 (m), 2869 (m), 1730 (m), 1713 (s), 1467 (w), 1401 (w), 1382 (m), 1365 (m), 1236 (s), 1150 (m), 1106 (m), 1034 (s), 1012 (s), 967 (m), 940 (m), 920 (m), 904 (m), 871 (w), 834 (w), 734 (w), 664 (w), 941 (w), 609 (w), 553 (w).

### Step f: Synthese von 3β-acetoxy-7α-bromo-5α,6α-dihydroxy-6β,19-epoxy-cholestan (7)

To a solution of 22.3 g (42.8 mmol, 1.0 eq.) of compound 6 in 400 mL of acetic acid was added 6.6 mL (20.5 g, 128 mmol, 3.5 eq.) of bromine in acetic acid (65 mL) and a few drops of HBr (48% aqu.). The reaction mixture was heated at 60° C. for 5 h. Subsequently, the reaction mixture was cooled to room temperature, and quenched with a saturated Na₂SO₃ solution. The aqueous phase was extracted with EtOAc, the combined organic phases were washed with water and a saturated NaCl solution, and dried over MgSO₄. Then, the solvent was removed under reduced pressure, and the product 7 was obtained after purification by column chromatography (c-hex/EtOAc, 6:1) in the form of a white solid (13.4 g, 24.2 mol, 57%).

Melting point: 124° C.-125° C.

¹H NMR (500 MHz, CDCl₃): δ [ppm]=5.05-5.00 (m, 1H), 4.12 (d, J=4.9 Hz, 1H), 4.02 (d, J=9.0 Hz, 1H), 3.73 (d, J=9.0 Hz, 1H), 3.09 (s, 1H), 2.89 (s, 1H), 2.19 (ddd, J=12.9, 4.5, 2.2 Hz, 1H), 2.04 (s, 3H), 2.05-1.99 (m, 1H), 1.97-1.83 (m, 5H), 1.74 (t, J=12.4 Hz, 1H), 1.62 (s, 2H), 1.59-0.98 (m, 19H), 0.91 (d, J=6.5 Hz, 3H), 0.87 (dd, J=6.6, 3.0 Hz, 6H), 0.74 (s, 3H).

¹³C NMR (125 MHz, CDCl₃): δ [ppm]=170.7, 101.7, 79.4, 69.9, 66.8, 59.4, 55.7, 52.9, 45.4, 43.5, 39.6, 39.0, 38.7, 38.6, 36.2, 35.8, 35.0, 28.2, 28.1, 27.2, 24.0, 24.0, 23.3, 23.0, 22.7, 21.6, 21.5, 18.8, 13.1.

FT-IR (ATR): ν [cm⁻¹]=3418 (br w), 2933 (m), 2868 (m), 1726 (s), 1714 (s), 1498 (w), 1466 (w), 1382 (m), 1366 (m), 1244 (s), 1154 (m), 1131 (m), 1096 (m), 1042 (s), 984 (m), 965 (s), 942 (s), 906 (m), 846 (m), 814 (w), 727 (w), 703 (m), 680 (m), 666 (m), 629 (m), 609 (m), 583 (m).

### Step g: Synthesis of 3β-acetoxy-5α-hydroxy-7β,19-epoxy-cholestan-6-one (8) (WIL-232)

22.6 g (40.7 mmol, 1.0 eq.) of compound 7 was dissolved in 1.8 L of dimethylformamide, and admixed with 38.3 g (0.52 mol, 12.7 eq.) Li₂CO₃ and 16.3 g (0.19 mol, 4.6 eq.) of LiBr. The reaction mixture was stirred at 100° C. for 2 h. Subsequently, the reaction mixture was cooled to room temperature, and diluted with water. The aqueous phase was extracted with EtOAc, the combined organic phases were washed with water and a saturated NaCl solution, and dried over MgSO₄. Subsequently, the solvent was removed under reduced pressure, and the product 8 was obtained after purification by column chromatography (c-hex/EtOAc, 6:1) in the form of a white solid (17.5 g, 37.0 mol, 91%).

Melting point: 146° C.-147° C.

¹H NMR (500 MHz, CDCl₃): δ [ppm]=5.25-5.18 (m, 1H), 4.14 (dd, J=10.2, 1.7 Hz, 1H), 3.91 (d, J=10.2 Hz, 1H), 3.82 (d, J=1.7 Hz, 1H), 2.61 (s, 1H), 2.12-2.03 (m, 3H), 2.03 (s, 3H), 2.00-1.94 (m, 2H), 1.86 (dt, J=13.0, 9.4, 5.8 Hz, 1H), 1.73 (td, J=13.8, 4.7 Hz, 1H), 1.65-1.48 (m, 5H), 1.42-1.04 (m, 13H), 0.98 (dt, J=10.2, 8.7 Hz, 1H), 0.89 (d, J=6.5 Hz, 3H), 0.86 (dd, J=6.6, 2.4 Hz, 6H), 0.74 (s, 3H).

¹³C NMR (125 MHz, CDCl₃): δ [ppm]=212.9, 170.5, 78.7, 76.2, 69.2, 63.6, 55.7, 52.0, 45.5, 45.1, 43.2, 40.5, 40.2, 39.6, 36.2, 35.8, 35.2, 28.6, 28.1, 26.2, 23.9, 22.9, 22.9, 22.7, 21.7, 21.5, 21.3, 18.7, 13.1.

FT-IR (ATR): ν [cm⁻¹]=3507 (br w), 2944 (s), 2934 (s), 2870 (m), 1734 (m), 1713 (s), 1469 (m), 1444 (m), 1381 (m), 1370 (m), 1254 (s), 1245 (vs), 1029 (vs), 977 (m), 961 (m), 911 (w), 886 (w), 824 (w), 807 (w), 742 (w), 670 (w), 610 (w).

HR-MS: (ESI, 70 eV)=m/z calculated for: C₂₉H₄₆O₅Na⁺ [M+Na]⁺ 497.32375 u, found: 497.32341 u.

### Step h: Synthesis of 3β,5α-dihydroxy-7β,19-epoxy-cholestan-6-one (9) (WIL-071)

To a solution of 12.7 g (26.7 mmol, 1.0 eq.) of 8 in 890 mL of methanol was added 5.5 g (40.1 mmol, 1.5 eq.) of K₂CO₃, and the reaction mixture was stirred at room temperature for 1 h. After the reaction was complete (TLC), the reaction mixture was diluted with water. The aqueous phase was extracted with EtOAc, the combined organic phases were washed with NaCl solution, and dried over MgSO₄. Subsequently, the solvent was removed under reduced pressure, and the residue was purified by column chromatography (c-hex/EtOAc, 1:1). The product 9 (WIL-071) was obtained in the form of a white solid (11.3 g, 26.1 mol, 98%), and subsequently recrystallized from about 400 mL (EtOH/H₂O, 1:1) (9.7 g of highly pure substance).

Melting point: 178° C.-180° C.

¹H NMR (600 MHz, CDCl₃): δ [ppm]=4.19-4.13 (m, 2H), 3.88 (d, J=10.2 Hz, 1H), 3.81 (s, 1H), 2.71 (s, 1H), 2.11-2.00 (m, 3H), 1.96-1.82 (m, 3H), 1.75 (s, 1H), 1.65-0.94 (m, 20H), 0.88 (d, J=6.5 Hz, 3H), 0.85 (dd, J=6.6, 2.9 Hz, 6H), 0.73 (s, 3H).

¹³C NMR (150 MHz, CDCl₃): δ [ppm]=213.6, 79.2, 76.2, 66.2, 63.7, 55.7, 52.1, 45.5, 45.2, 43.3, 40.6, 40.3, 39.6, 38.8, 36.2, 35.8, 30.2, 28.6, 28.1, 23.9, 22.9, 22.9, 22.7, 21.8, 21.5, 18.7, 13.1.

FT-IR (ATR): ν [cm⁻¹]=3463 (br w), 2948 (s), 2868 (m), 1733 (vs), 1497 (w), 1466 (m), 1414 (w), 1382 (m), 1365 (w), 1340 (w), 1296 (w), 1248 (m), 1225 (w), 1153 (m), 1051 (vs), 968 (m), 958 (m), 887 (w), 818 (w), 779 (w), 756 (m), 713 (w), 637 (w), 558 (m), 539 (w).

HR-MS: (ESI, 70 eV)=m/z calculated for: C₂₇H₄₄O₄Na⁺ [M+Na]⁺ 455.31318 u, found: 455.31365 u.

### Synthesis of 5α-hydroxy-7β,19-epoxy-cholestane-3,6-dione (10) (WIL-241)

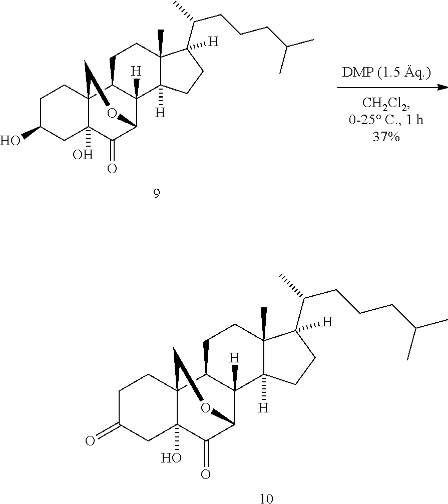

To a solution of 400 mg (0.93 mmol, 1.0 eq.) of alcohol 9 in 11.5 mL of dichloromethane at 0° C. was added 588 mg (1.39 mmol, 1.5 eq.) of Dess-Martin periodinane, and the reaction mixture was stirred at room temperature for 1 h. After the reaction was complete (TLC), the reaction mixture was diluted with aqueous NaHCO₃ solution and aqueous Na₂S₂O₃ solution. The aqueous phase was extracted with CH₂Cl₂, the combined organic phases were dried over MgSO₄, and the solvent was removed under reduced pressure. The residue was purified by column chromatography (chex/EtOAc, 5:1), and the product 10 was obtained in the form of a white solid (148 mg, 0.34 mmol, 37%).

Melting point: 204° C.-205° C.

¹H NMR (500 MHz, CDCl₃): δ [ppm]=4.12 (q, J=10.5 Hz, 2H), 3.93 (d, J=1.6 Hz, 1H), 2.99 (d, J=15.6 Hz, 1H), 2.92-2.87 (m, 1H), 2.48 (m, J=16.3, 8.0 Hz, 2H), 2.43-2.34 (m, 1H), 2.19 (t, J=10.6 Hz, 1H), 2.11-2.00 (m, 2H), 1.93-1.84 (m, 1H), 1.65-1.56 (m, 3H), 1.55-1.06 (m, 14H), 1.03-0.95 (m, 1H), 0.90 (d, J=6.5 Hz, 3H), 0.86 (dd, J=6.6, 2.5 Hz, 6H), 0.77 (s, 3H).

¹³C NMR (126 MHz, CDCl₃): δ [ppm]=211.5, 206.5, 80.0, 76.8, 63.6, 55.7, 51.9, 47.1, 45.4, 45.1, 43.4, 40.9, 40.2, 39.6, 36.9, 36.2, 35.8, 28.6, 28.1, 23.9, 22.9, 22.9, 22.7, 22.4, 21.8, 18.7, 13.1.

FT-IR (ATR): ν [cm⁻¹]=3459 (bw), 2952 (m), 2940 (m), 2918 (m), 2859 (m), 1742 (s), 1714 (s), 1466 (m), 1382 (m), 1345 (w), 1298 (w), 1252 (w), 1238 (m), 1195 (w), 1163 (m), 1145 (m), 1102 (w), 1079 (w), 1051 (s), 980 (w), 948 (m), 913 (w), 903 (w), 891 (w), 854 (w), 822 (w), 797 (w), 752 (m), 723 (m), 663 (w), 631 (m), 594 (w), 559 (m), 525 (m).

HR-MS: (ESI, 70 eV)=m/z calculated for: C₂₇H₄₃O₄⁺ [M+H]⁺ 431.31559 u, found: 431.31544 u;

m/z calculated for: C₂₇H₄₂O₄Na⁺ [M+Na]⁺ 453.29753 u, found: 453.29761 u.

### Synthesis of 7β,19-epoxy-cholest-4-en-3,6-dione (11) (WIL-270)

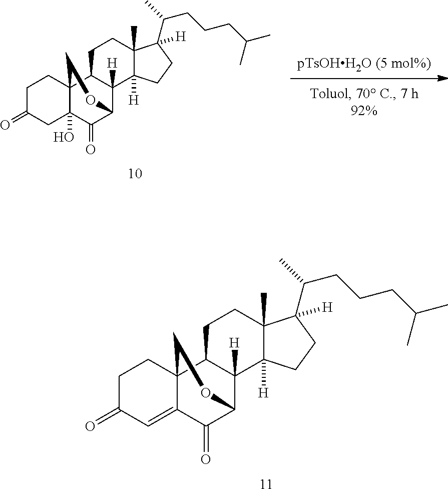

To a solution of 50 mg (0.12 mmol, 1.0 eq.) of 10 in 1.2 mL of toluene was added 1.8 mg (5.8 mmol, 0.05 eq.) of pTsOH-H₂O at room temperature. The reaction mixture was heated at 70° C. and stirred at 70° C. for 7 h. After the reaction was complete (TLC), the reaction mixture was diluted with CH₂Cl₂ and diluted aqueous NaHCO₃ solution. The aqueous phase was extracted with CH₂Cl₂, the combined organic phases were dried over MgSO₄, and the solvent was removed under reduced pressure. The residue was purified by column chromatography (chex/EtOAc, 6:1). The product 11 was obtained in the form of a white solid (44 mg, 0.11 mmol, 92%).

Melting point: 127° C.-128° C.

¹H NMR (500 MHz, CDCl₃): δ [ppm]=6.42 (s, 1H), 4.03 (d, J=8.9 Hz, 1H), 3.95 (s, 1H), 3.91 (d, J=8.9 Hz, 1H), 2.50-2.43 (m, 2H), 2.23 (t, J=10.9 Hz, 1H), 2.13 (dt, J=13.2, 3.1 Hz, 1H), 2.06-1.96 (m, 1H), 1.95-1.83 (m, 2H), 1.70-1.62 (m, 1H), 1.62-1.47 (m, 3H), 1.44-1.06 (m, 12H), 1.03-0.96 (m, 1H), 0.90 (d, J=6.5 Hz, 3H), 0.86 (dd, J=6.6, 2.4 Hz, 6H), 0.78 (s, 3H)

¹³C NMR (126 MHz, CDCl₃): δ [ppm]=198.6, 197.7, 156.4, 122.7, 77.0, 64.3, 55.7, 52.4, 49.3, 45.4, 43.5, 40.3, 39.6, 38.4, 36.2, 35.8, 34.7, 28.7, 28.1, 24.2, 23.9, 22.9, 22.9, 22.7, 21.9, 18.7, 13.1.

FT-IR (ATR): ν [cm⁻¹]=2954 (m), 2933 (m), 2857 (m), 1724 (m), 1688 (s), 1632 (w), 1465 (m), 1453 (m), 1383 (w), 1366 (w), 1328 (w), 1274 (w), 1245 (w), 1231 (m), 1185 (m), 1113 (w), 1074 (w), 1035 (s), 974 (w), 932 (w), 904 (s), 879 (w), 857 (w), 828 (m), 811 (w), 780 (m), 766 (w), 720 (w), 653 (w), 634 (w), 571 (w), 548 (m), 529 (m), 507 (w).

HR-MS: (ESI, 70 eV)=m/z calculated for: C₂₇H₄₁O₃⁺ [M+H]⁺ 413.30502 u, found: 413.30513 u;

m/z calculated for: C₂₇H₄₀O₃Na⁺ [M+Na]⁺ 435.28697 u, found: 435.28720 u.

### Synthesis of 3β,5α-dibenzyloxy-7β,19-epoxy-cholestan-6-one (12) (WIL 242a) and of 5α-benzyloxy-3β-hydroxy-7β,19-epoxy-cholestan-6-one (13) (WIL 242b)

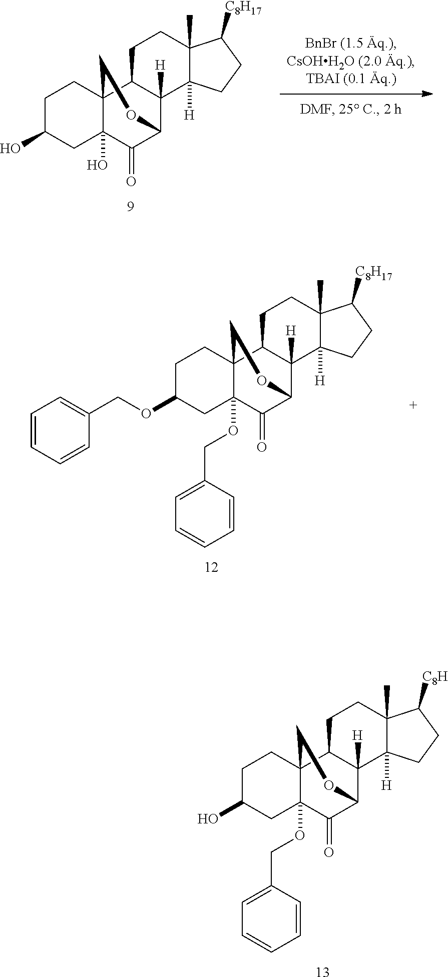

To a solution of 39 mg (0.090 mmol, 1.0 eq.) of 9 in 0.92 mL of dimethylformamide were added 35 mg (0.185 mmol, 2.0 eq.) of CsOH·H₂O, 16.2 mL (0.139 mmol, 1.5 eq.) of BnBr and 4 mg (0.011 mmol, 0.1 eq.) of tetrabutylammonium iodide at room temperature. The reaction mixture was stirred at room temperature for 2 h. After the reaction was complete (TLC), the reaction mixture was diluted with MTBE and diluted aqueous NH₄Cl solution. The aqueous phase was extracted with MTBE, the combined organic phases were washed first with water, then with saturated NaCl solution, subsequently dried over MgSO₄, and the solvent was removed under reduced pressure. The residue was purified by column chromatography (chex/EtOAc, 9:1 to 3:1). The product 12 (doubly Bn) was obtained in the form of a colorless oil (22 mg, 0.036 mmol, 40%). In addition, the product 13 (singly benzylated) was also obtained in the form of a colorless oil (7.9 mg, 0.015 mmol, 17%).

Analytics relating to 12:

¹H NMR (500 MHz, CDCl₃): δ [ppm]=7.38-7.25 (m, 5H), 5.15 (d, J=11.9 Hz, 1H), 4.79 (d, J=12.0 Hz, 1H), 4.13 (d, J=10.3 Hz, 1H), 3.88 (d, J=10.2 Hz, 2H), 3.67 (s, 1H), 2.65 (dd, J=14.2, 3.5 Hz, 1H), 2.09-1.97 (m, 3H), 1.96-1.73 (m, 4H), 1.63-1.06 (m, 18H), 1.02-0.95 (m, 1H), 0.89 (d, J=6.5 Hz, 3H), 0.86 (dd, J=6.6, 2.5 Hz, 6H), 0.74 (s, 3H).

¹³C NMR (126 MHz, CDCl₃): δ [ppm]=208.4, 139.3, 128.4, 127.1, 80.7, 66.1, 65.5, 63.7, 55.7, 52.6, 45.6, 44.1, 43.0, 41.4, 40.6, 39.6, 36.3, 35.8, 33.2, 30.5, 28.7, 28.2, 23.9, 23.0, 22.9, 22.7, 22.0, 21.9, 18.7, 13.1.

HR-MS: (ESI, 70 eV)=m/z calculated for: C₃₄H₅₀O₄Na⁺ [M+Na]⁺545.36013 u, found: 545.36016 u.

Analytics relating to 13:

¹H NMR (500 MHz, CDCl₃): δ [ppm]=7.38-7.23 (m, 8H), 7.16 (d, J=6.9 Hz, 2H), 5.07 (d, J=11.6 Hz, 1H), 4.58-4.51 (m, 3H), 4.15 (d, J=9.9 Hz, 1H), 3.86 (d, J=10.1 Hz, 1H), 3.66 (s, 1H), 3.64-3.55 (m, 1H), 2.72 (dd, J=14.2, 3.4 Hz, 1H), 2.08-1.93 (m, 4H), 1.91-1.83 (m, 1H), 1.79 (dd, J=14.1, 11.4 Hz, 1H), 1.72 (td, J=13.8, 4.7 Hz, 1H), 1.62-1.46 (m, 4H), 1.41-1.06 (m, 13H), 1.02-0.94 (m, 1H), 0.89 (d, J=6.4 Hz, 3H), 0.86 (dd, J=6.6, 2.5 Hz, 6H), 0.73 (s, 3H).

¹³C NMR (126 MHz, CDCl₃): δ [ppm]=208.7, 139.2, 138.7, 128.6, 128.4, 127.8, 127.3, 127.3, 80.6, 72.3, 70.6, 65.5, 63.7, 55.8, 52.6, 45.6, 44.1, 43.1, 41.6, 40.6, 39.6, 36.3, 35.8, 30.2, 28.7, 28.2, 27.3, 23.9, 23.0, 23.0, 22.7, 21.9, 21.8, 18.7, 13.1, 0.2.

FT-IR (ATR): ν [cm⁻¹]=3064 (w), 3031 (w), 2949 (s), 2867 (s), 1733 (s), 1496 (w), 1454 (m), 1382 (w), 1364 (w), 1307 (w), 1262 (w), 1228 (w), 1206 (w), 1179 (w), 1142 (w), 1090 (s), 1054 (m), 1001 (w), 953 (w), 873 (w), 842 (w), 803 (w), 734 (s), 693 (s), 557 (w).

HR-MS: (ESI, 70 eV)=m/z calculated for: C₄₁H₅₆O₄Na⁺ [M+Na]⁺635.40708 u, found: 635.40751 u.

### Synthesis of (7β,19-epoxy-5α-hydroxy-6-keto-cholestan-3β-yl) ester of propionic acid (14) [WIL-313]

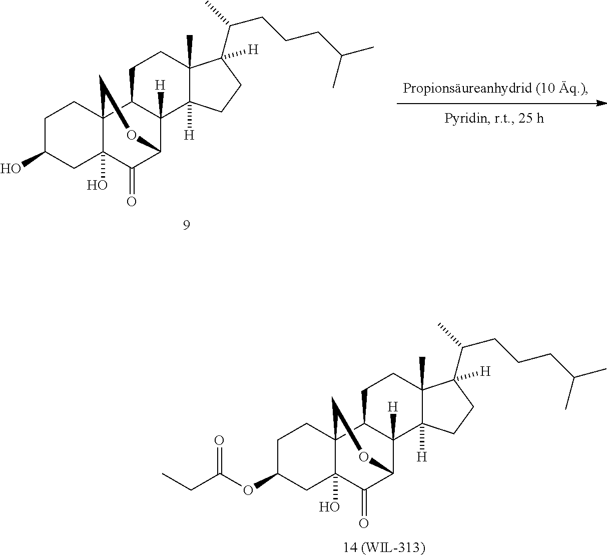

To a solution of 100 mg (0.232 mmol, 1.0 eq.) of alcohol 9 in 0.58 mL of pyridine was added 0.30 ml (2.32 mmol, 10 eq.) propionic anhydride at room temperature. The reaction mixture was stirred at room temperature for 25 h, and after the reaction was complete (TLC), it was diluted with ethyl acetate. The organic phase was washed twice with 1 M hydrochloric acid, and once with water, and subsequently dried over MgSO₄. The solvent was removed under reduced pressure, and the residue was purified by column chromatography on silica (cHex/EtOAc, 10:1). The product 14 could be obtained in the form of a white solid (92 mg, 0.19 mmol, 81%).

Analytics relating to 14:

Melting point: 50° C.-55° C.

Rf: 0.50 (3:1, cHex:EtOAc).

¹H NMR: (500 MHz, CDCl₃) δ [ppm]: 5.23 (tt, J=11.1, 5.5 Hz, 1H), 4.19-4.11 (m, 1H), 3.91 (d, J=10.2 Hz, 1H), 3.82 (d, J=1.5 Hz, 1H), 2.61 (s, 1H), 2.29 (q, J=7.5 Hz, 2H), 2.13-2.03 (m, 3H), 2.00-1.93 (m, 2H), 1.91-1.82 (m, 1H), 1.73 (td, J=13.9, 4.7 Hz, 1H), 1.65-1.47 (m, 4H), 1.44-1.07 (m, 17H), 1.02-0.95 (m, 1H), 0.89 (d, J=6.6 Hz, 3H), 0.86 (dd, J=6.7, 2.4 Hz, 6H), 0.74 (s, 3H).

¹³C NMR: (126 MHz, CDCl₃) δ [ppm]: (126 MHz, CDCl₃): δ [ppm]=212.89, 173.96, 78.67, 76.18, 68.93, 63.57, 55.73, 52.03, 45.52, 45.09, 43.22, 40.53, 40.24, 39.59, 36.22, 35.77, 35.27, 28.64, 28.13, 28.00, 26.24, 23.88, 22.93, 22.89, 22.69, 21.71, 21.27, 18.74, 13.07, 9.30.

FT-IR (ATR): ν [cm⁻¹]=3444 (bw), 2947 (m), 2868 (m), 1737 (s), 1463 (m), 1381 (m), 1350 (m), 1275 (w), 1261 (w), 1181 (s), 1155 (m), 1082 (m), 1046 (m), 1022 (m), 975 (w), 952 (m), 889 (w), 869 (m), 824 (w), 808 (w), 757 (m), 711 (w), 663 (w), 637 (w), 559 (m).

HR-MS: (GC-EI-MS, 70 eV)=m/z calculated for: C₃₀H₄₈O₅[M]⁺ 488.34963 u, found: 488.34896 u.

[α]_{λ}²⁰: c=0.555 g/100 mL, CHCl₃:
[α]₃₆₅²⁰=108.83° (±0.15°),
[α]₄₃₆²⁰=64.44° (±0.22°),
[α]₅₄₆²⁰=37.48° (±0.00°),
[α]₅₇₉²⁰=32.43° (±0.15°),
[α]₅₈₉²⁰=30.45° (±0.00°).

### Synthesis of 7β,19-epoxy-5α-hydroxy-6-on-cholestan-3β-yl-methyl carbonate (15) [WIL-357]

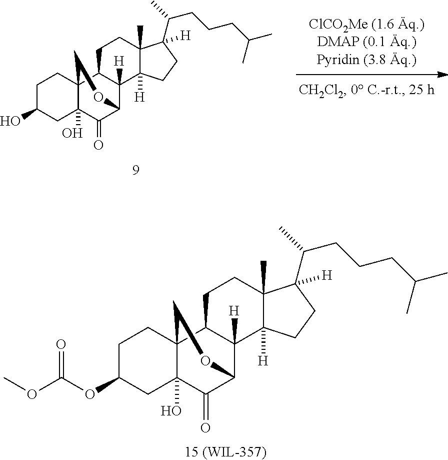

To a solution of 99 mg (0.231 mmol, 1.0 eq.) of alcohol 9 in 0.49 mL of dichloromethane were added 0.07 ml (8.8 mmol, 3.8 eq.) of pyridine, 3 mg (0.025 mmol, 0.1 eq.) of DMAP and 0.03 (0.38 mmol, 1.6 eq.) of methyl chloroformate at 0° C. After 1.5 h at 0° C., the reaction mixture was heated at room temperature, and stirred at room temperature for 24 h. After the reaction was complete (TLC), the reaction mixture was diluted with an aqueous saturated NaCl solution and water. The aqueous phase was extracted three times with dichloromethane, and the combined organic phases were washed with 1 M hydrochloric acid, and with saturated aqueous NaCl solution, and subsequently dried over MgSO₄. The solvent was removed under reduced pressure, and the residue was purified by column chromatography on silica (cHex/EtOAc, 10:1). The product 15 could be obtained in the form of a white solid (93 mg, 0.19 mmol, 81%).

Analytics relating to 15:

Melting point: 64° C.-65° C.

Rf: 0.67 (1:1, cHex:EtOAc).

¹H NMR: (500 MHz, CDCl₃) δ [ppm]: 5.09 (tt, J=11.1, 5.4 Hz, 1H), 4.12 (dd, J=10.4, 1.6 Hz, 1H), 3.90 (d, J=10.3 Hz, 1H), 3.82 (d, J=1.7 Hz, 1H), 3.77 (s, 3H), 2.63 (s, 1H), 2.17 (ddd, J=13.0, 5.2, 1.6 Hz, 1H), 2.13-1.99 (m, 4H), 1.86 (dtd, J=13.1, 9.5, 5.9 Hz, 1H), 1.73 (td, J=13.7, 4.5 Hz, 1H), 1.66-1.55 (m, 3H), 1.51 (dt, J=13.2, 6.6 Hz, 1H), 1.43-1.07 (m, 14H), 1.02-0.95 (m, 1H), 0.89 (d, J=6.5 Hz, 3H), 0.86 (dd, J=6.6, 2.5 Hz, 6H), 0.74 (s, 3H).

¹³C NMR: (126 MHz, CDCl₃) δ [ppm]: 212.72, 155.19, 78.65, 76.12, 73.29, 63.51, 55.73, 54.82, 52.02, 45.52, 45.09, 43.20, 40.51, 40.24, 39.59, 36.22, 35.76, 35.21, 28.64, 28.13, 26.18, 23.88, 22.93, 22.88, 22.69, 21.71, 21.22, 18.74, 13.08.

FT-IR (ATR): ν [cm⁻¹]=3457 (bw), 2950 (m), 2867 (w), 1743 (s), 1443 (m), 1383 (w), 1320 (w), 1269 (s), 1258 (s), 1227 (m), 1171 (w), 1154 (w), 1046 (m), 1018 (m), 952 (m), 935 (m), 877 (w), 825 (w), 792 (m), 757 (w), 721 (w), 664 (w), 625 (w), 559 (w), 530 (w).

HR-MS: (GC-EI-MS, 70 eV)=m/z calculated for: C₂₇H₄₆O₆[M]⁺ 490.32889 u, found: 490.32834 u.

[α]_{λ}²⁰: c=0.58 g/100 mL, CHCl₃:
[α]₃₆₅²⁰=105.11° (±0.08°),
[α]₄₃₆²⁰=61.21° (±0.00°),
[α]₅₄₆²⁰=35.34° (±0.00°),
[α]₅₇₉²⁰=30.46° (±0.08°),
[α]₅₈₉²⁰=28.79° (±0.00°),

### Synthesis of N-Boc-glycin (7β,19-epoxy-5α-hydroxy-6-on-cholestan-3β-yl) ester (16) [WIL-360]

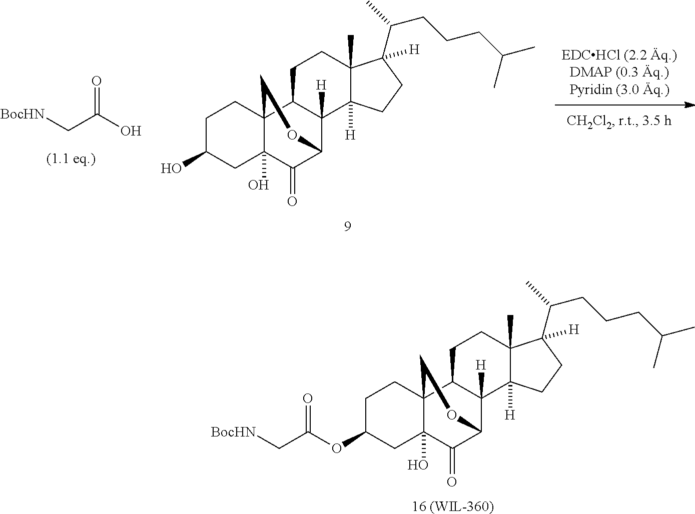

To a solution of 199 mg (0.460 mmol, 1.0 eq.) of alcohol 9 in 4.6 mL of dichloromethane were added 0.12 ml (1.5 mmol, 3.4 eq.) of pyridine, 195 mg (1.02 mmol, 2.2 eq.) of EDC·HCl, 18 mg (0.14 mmol, 0.3 eq.) of DMAP and 89 mg (0.51 mmol, 1.1 eq.) of N-Boc-glycine under an argon atmosphere. The reaction mixture was stirred at room temperature for 3.5 h. After the reaction was complete (TLC), the reaction mixture was diluted with 20 ml of ethyl acetate. The organic phase was washed once each with saturated aqueous NaHCO₃ solution, water and saturated aqueous NaCl solution. The organic phase was subsequently dried over MgSO₄, and the solvent was removed under reduced pressure. The residue was purified by column chromatography on silica (10:1, cHex/EtOAc), and the product 16 was obtained in the form of a colorless solid (262 mg, 0.444 mmol, 96%).

Analytics relating to 16:

Melting point: 76° C.-77° C.

Rf: 0.42 (3:1, cHex:EtOAc).

¹H NMR: (500 MHz, CDCl₃) δ [ppm]: 5.29 (tt, J=11.1, 5.5 Hz, 1H, H-3), 5.04-4.71 (m, 1H, NH), 4.12 (d, J=10.8 Hz, 1H, H-19), 3.94-3.78 (m, 4H, H-29, H-19, H-7), 2.64 (s, 1H, OH), 2.12-1.95 (m, 5H), 1.91-1.81 (m, 1H), 1.73 (td, J=13.7, 4.6 Hz, 1H), 1.63-1.54 (m, 4H), 1.50 (dt, J=13.3, 6.6 Hz, 1H), 1.45 (s, 9H), 1.41-1.05 (m, 13H), 0.98 (q, J=9.1 Hz, 1H), 0.89 (d, J=6.6 Hz, 3H), 0.85 (dd, J=6.6, 2.4 Hz, 6H), 0.74 (s, 3H).

¹³C NMR: (126 MHz, CDCl₃) δ [ppm]: 212.73, 169.86, 155.81, 80.13, 78.62, 76.15, 70.50, 63.50, 55.73, 52.01, 45.51, 45.08, 43.20, 42.75, 40.51, 40.23, 39.59, 36.21, 35.76, 35.19, 28.63, 28.46, 28.13, 26.15, 23.88, 22.93, 22.88, 22.69, 21.69, 21.21, 18.74, 13.07.

FT-IR (ATR): □ [cm⁻¹]=3387 (bw), 2951 (m), 2869 (m), 1740 (s), 1720 (s), 1513 (m), 1467 (w), 1384 (m), 1366 (m), 1282 (w), 1251 (m), 1204 (m), 1169 (s), 1051 (m), 970 (w), 918 (w), 867 (w), 825 (w), 757 (w), 734 (w), 559 (w).

HR-MS: (ESI, 70 eV)=m/z calculated for: C₃₄H₅₅NO₇Na [M+Na]⁺612.38707 u, found: 612.38748 u.

[α]_{λ}²⁰: c=0.575 g/100 mL, CHCl₃:
[α]₃₆₅²⁰=94.90° (±0.22°),
[α]₄₃₆²⁰=56.87° (±0.00°),
[α]₅₃₆²⁰=33.39° (±0.00°),
[α]₅₇₉²⁰=28.98° (±0.08°),
[α]₅₈₉²⁰=27.42° (±0.08°),

### Synthesis of (7β,19-epoxy-5α-hydroxy-6-on-cholestan-3β-yl) ester of (tert-butyldimethylsilanyloxy)acetic acid (17) [WIL-361]

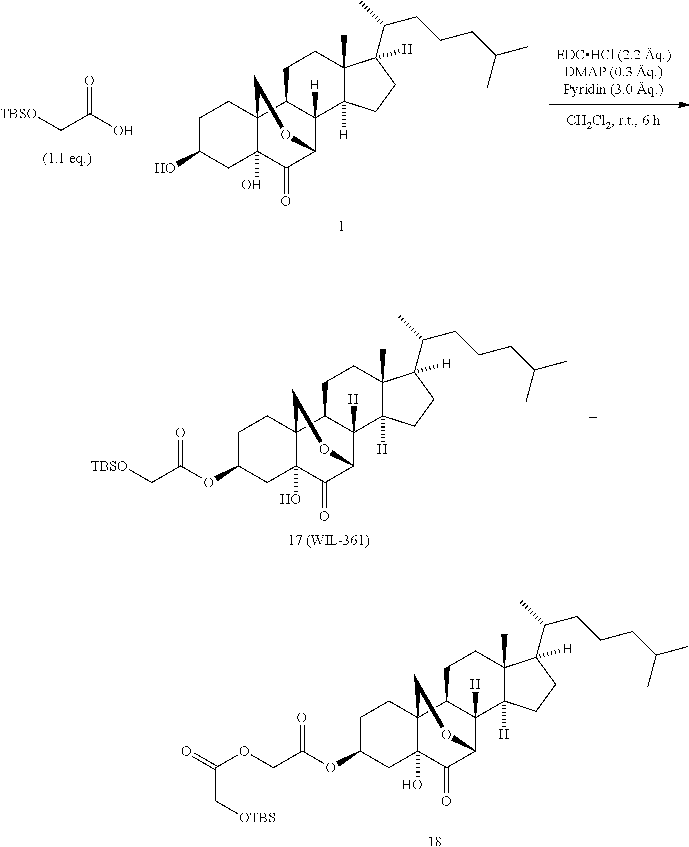

To a solution of 198 mg (0.458 mmol, 1.0 eq.) of alcohol 1 in 4.6 mL of dichloromethane were added 0.12 ml (1.5 mmol, 3.4 eq.) of pyridine, 197 mg (1.02 mmol, 2.2 eq.) of EDC·HCl, 18 mg (0.14 mmol, 0.3 eq.) of DMAP and 98 mg (0.51 mmol, 1.1 eq.) of TBS-glycolic acid under an argon atmosphere. The reaction mixture was stirred at room temperature for 6 h. After the reaction was complete (TLC), the reaction mixture was diluted with 20 ml of ethyl acetate. The organic phase was washed once each with saturated aqueous NaHCO₃ solution, water and saturated aqueous NaCl solution. The organic phase was subsequently dried over MgSO₄, and the solvent was removed under reduced pressure. The residue was purified by column chromatography on silica (20:1, cHex/EtOAc), and the desired product 17 could be obtained in the form of a colorless solid (165 mg, 0.273 mmol, 59%). In addition, the undesirable by-product 18 could also be obtained as a colorless solid (53 mg, 0.080 mmol, 17%).

Analytics relating to 17:

Melting point: 139° C.-140° C.

Rf: 0.59 (3:1, cHex:EtOAc).

¹H NMR: (500 MHz, CDCl₃) δ [ppm]: 5.29 (dt, J=11.3, 5.6 Hz, 1H), 4.20 (s, 2H), 4.12 (d, J=9.3 Hz, 1H), 3.90 (d, J=10.2 Hz, 1H), 3.81 (d, J=1.5 Hz, 1H), 2.60 (s, 1H), 2.13-1.94 (m, 5H), 1.91-1.81 (m, 1H), 1.73 (td, J=13.9, 4.7 Hz, 1H), 1.64-1.46 (m, 4H), 1.43-1.06 (m, 14H), 1.02-0.95 (m, 1H), 0.91 (s, 9H), 0.89 (d, J=6.6 Hz, 3H), 0.85 (dd, J=6.6, 2.4 Hz, 6H), 0.74 (s, 3H), 0.09 (d, J=1.3 Hz, 6H).

¹³C NMR: (151 MHz, CDCl₃) δ [ppm]: 212.74, 171.29, 78.62, 76.15, 69.83, 63.54, 62.08, 55.73, 52.02, 45.52, 45.07, 43.20, 40.52, 40.23, 39.59, 36.21, 35.77, 35.20, 28.64, 28.13, 26.19, 25.88, 23.88, 22.93, 22.89, 22.69, 21.70, 21.24, 18.74, 18.54, 13.07, −5.25, −5.27.

FT-IR (ATR): ν [cm⁻¹]=3494 (w), 2952 (m), 2931 (m), 2896 (w), 2858 (w), 1741 (m), 1495 (w), 1463 (w), 1441 (w), 1383 (w), 1361 (w), 1252 (m), 1221 (m), 1187 (w), 1147 (s), 1085 (w), 1048 (m), 1021 (m), 977 (w), 961 (w), 888 (w), 835 (s), 780 (s), 755 (m), 712 (m), 663 (w), 636 (w), 602 (w), 559 (w), 474 (w), 434 (w).

HR-MS: (ESI, 70 eV)=m/z calculated for: C₃₅H₆₀O₆SiNa [M+Na]⁺627.40514 u, found: 627.40559 u.

[α]_{λ}²⁰: c=0.53 g/100 mL, CHCl₃:
[α]₃₆₅²⁰=97.86° (±0.09°),
[α]₄₃₆²⁰=58.55° (±0.09°),
[α]₅₄₆²⁰=34.47° (±0.18°),
[α]₅₇₉²⁰=30.25° (±0.35°),
[α]₅₈₉²⁰=28.30° (±0.16°),

Analytics relating to 18:

Melting point: 170° C.-171° C.

Rf: 0.48 (3:1, cHex:EtOAc).

¹H NMR: (500 MHz, CDCl₃) δ [ppm]: 5.33 (tt, J=11.1, 5.5 Hz, 1H), 4.69-4.60 (m, 2H), 4.38 (s, 2H), 4.14 (dd, J=10.3, 1.8 Hz, 1H), 3.93 (d, J=10.2 Hz, 1H), 3.84 (d, J=1.7 Hz, 1H), 2.63 (s, 1H), 2.15-1.98 (m, 5H), 1.93-1.84 (m, 1H), 1.75 (td, J=13.8, 4.7 Hz, 1H), 1.65-1.49 (m, 4H), 1.44-1.08 (m, 14H), 1.04-0.97 (m, 1H), 0.94 (s, 9H), 0.91 (d, J=6.5 Hz, 3H), 0.88 (dd, J=6.6, 2.4 Hz, 6H), 0.76 (s, 3H), 0.14 (s, 6H).

¹³C NMR: (126 MHz, CDCl₃) δ [ppm]: 212.63, 171.31, 166.98, 78.59, 76.13, 70.83, 63.49, 61.59, 60.96, 55.73, 52.00, 45.51, 45.08, 43.19, 40.51, 40.22, 39.59, 36.21, 35.76, 35.14, 28.63, 28.13, 26.09, 25.88, 23.88, 22.93, 22.88, 22.69, 21.69, 21.20, 18.74, 18.54, 13.07, −5.31.

FT-IR (ATR): ν [cm⁻¹]=3523 (w), 2950 (m), 2930 (m), 2885 (w), 2858 (w), 1771 (m), 1754 (m), 1739 (m), 1470 (w), 1444 (w), 1389 (w), 1367 (w), 1255 (w), 1223 (w), 1180 (m), 1141 (s), 1040 (m), 1018 (w), 975 (w), 956 (w), 891 (w), 880 (w), 836 (s), 782 (m), 760 (m), 729 (w), 693 (w), 663 (w), 635 (w), 561 (w), 545 (w), 454 (w), 408 (w).

HR-MS: (ESI, 70 eV)=m/z calculated for: C₃₇H₆₂O₈SiNa [M+Na]⁺685.41062 u, found: 685.41025 u.

### Synthesis of (7β,19-epoxy-5α-hydroxy-6-on-cholestan-3β-yl) ester of glycolic acid (19) [WIL-364]

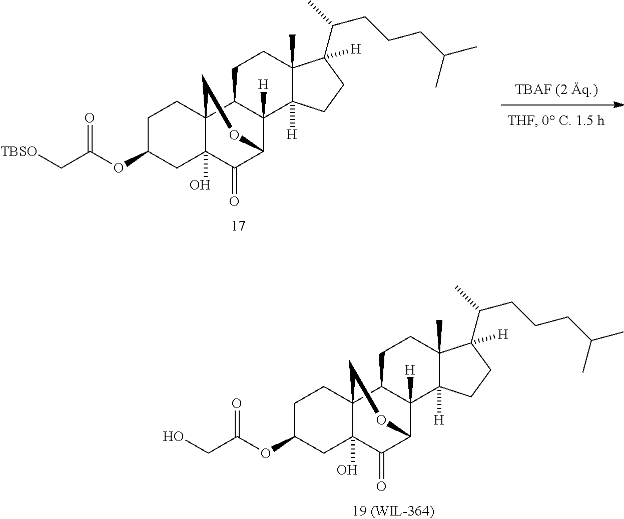

A solution of 104 mg (0.172 mmol, 1.0 eq.) of the TBSW ether 17 in 1.7 mL of THF was cooled to 0° C., and subsequently admixed with 0.34 ml (0.34 mmol, 2.0 eq.) of TBAF. After 1.5 h, and if the reaction was complete (TLC), the reaction mixture was admixed with a saturated aqueous NaCl solution. The aqueous phase was extracted three times with ethyl acetate, the combined organic phases were dried over MgSO₄, and the solvent was removed under reduced pressure. The residue was purified by column chromatography on silica (3:1, cHex/EtOAc), and the desired product 19 could be obtained in the form of a colorless solid (76 mg, 0.15 mmol, 90%).

Analytics relating to 19:

Melting point: 164° C.-166° C.

Rf: 0.17 (2:1, cHex:EtOAc).

¹H NMR: (500 MHz, CDCl₃) δ [ppm]: 5.35 (tt, J=11.2, 5.5 Hz, 1H), 4.14-4.08 (m, 3H), 3.90 (d, J=10.3 Hz, 1H), 3.81 (d, J=1.7 Hz, 1H), 2.79 (s, 1H), 2.47 (t, J=5.4 Hz, 1H), 2.14-2.02 (m, 3H), 2.02-1.96 (m, 2H), 1.85 (dtd, J=13.1, 9.4, 6.0 Hz, 1H), 1.74 (td, J=13.9, 4.7 Hz, 1H), 1.63-1.54 (m, 3H), 1.52-1.46 (m, 1H), 1.41-1.03 (m, 14H), 1.01-0.93 (m, 1H), 0.88 (d, J=6.5 Hz, 3H), 0.84 (dd, J=6.6, 2.9 Hz, 6H), 0.73 (s, 3H).

¹³C NMR: (126 MHz, CDCl₃) δ [ppm]: 212.75, 172.97, 78.58, 76.17, 70.93, 63.48, 60.88, 55.73, 52.00, 45.52, 45.06, 43.20, 40.51, 40.22, 39.59, 36.22, 35.76, 35.17, 28.63, 28.14, 26.18, 23.88, 22.93, 22.89, 22.69, 21.70, 21.22, 18.74, 13.07.

FT-IR (ATR): ν [cm⁻¹]=3454 (bw), 2949 (m), 1932 (m), 2867 (m), 1738 (s), 1465 (m), 1446 (m), 1383 (m), 1366 (w), 1223 (s), 1208 (s), 1155 (m), 1097 (s), 1087 (s), 1045 (s), 1020 (m), 973 (m), 961 (m), 920 (w), 888 (w), 824 (m), 780 (w), 757 (m), 716 (w), 663 (w), 637 (w), 559 (m), 522 (m), 477 (m), 431 (m).

HR-MS: (ESI, 70 eV)=m/z calculated for: C₂₉H₄₆O₆Na [M+Na]⁺ 513.31866 u, found: 513.31859 u.

[α]_{λ}²⁰: c=0.56 g/100 mL, CHCl₃:
[α]₃₆₅²⁰=95.59° (±0.30°),
[α]₄₃₆²⁰=57.98° (±0.08°),
[α]₅₄₆²⁰=34.17° (±0.22°),
[α]₅₇₉²⁰=30.12° (±0.22°),
[α]₅₈₉²⁰=28.21° (±0.14°),

### Synthesis of (7β,19-epoxy-5α-hydroxy-6-on-cholestan-3β-yl) ester of benzoic acid (20) [WIL-362]

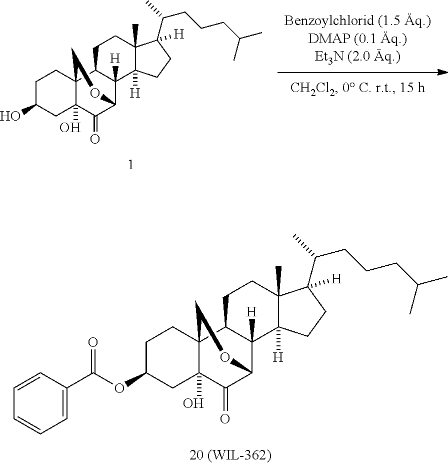

To a solution of 150 mg (0.347 mmol, 1.0 eq.) of alcohol 1, 6 mg (0.05 mmol, 0.1 eq.) of DMAP, 0.10 ml (0.69 mmol, 2.0 eq.) of triethylamine in 2.3 ml DCM was added 0.06 ml (0.52 mmol, 1.5 eq.) of benzoyl chloride at 0° C. under an argon atmosphere. After 15 min at 0° C., the reaction mixture was stirred at room temperature for another 15 h. After the reaction was complete (TLC), the reaction mixture was diluted with dichloromethane and water, and the aqueous phase was extracted three times with dichloromethane. The combined organic phases were washed with saturated aqueous NaCl solution, dried over MgSO₄, and subsequently, the solvent was removed under reduced pressure. The residue was purified by column chromatography on silica (40:1-15:1; cHex/EtOAc), and the desired product 20 could be obtained in the form of a colorless solid (158 mg, 0.294 mmol, 85%).

Analytics relating to 20:

Melting 151° C.-152° C.

point:

Rf: 0.55 (3:1, cHex:EtOAc).

¹H NMR: (500 MHz, CDCl₃) δ [ppm]: 8.07-8.01 (m, 2H), 7.61-7.54 (m, 1H), 7.45 (t, J=7.8 Hz, 2H), 5.51 (tt, J=11.0, 5.5 Hz, 1H), 4.25 (dd, J=10.3, 1.7 Hz, 1H), 3.97 (d, J=10.3 Hz, 1H), 3.87 (d, J=1.7 Hz, 1H), 2.70 (s, 1H), 2.25 (ddd, J=13.0, 5.3, 1.5 Hz, 1H), 2.19-2.07 (m, 4H), 1.95-1.78 (m, 2H), 1.78-1.59 (m, 3H), 1.53 (dt, J=13.1, 6.6 Hz, 1H), 1.48-1.09 (m, 14H), 1.06-0.98 (m, 1H), 0.92 (d, J=6.5 Hz, 3H), 0.88 (dd, J=6.6, 2.4 Hz, 6H), 0.77 (s, 3H).

¹³C NMR: (126 MHz, CDCl₃) δ [ppm]: 212.86, 166.01, 133.04, 130.61, 129.71, 128.46, 78.74, 76.21, 69.83, 63.62, 55.74, 52.04, 45.54, 45.12, 43.26, 40.61, 40.26, 39.60, 36.23, 35.78, 35.39, 28.66, 28.14, 26.35, 23.89, 22.94, 22.91, 22.70, 21.74, 21.33, 18.75, 13.09.

FT-IR (ATR): ν [cm⁻¹]=3423 (bw), 2949 (m), 2867 (m), 1739 (m), 1717 (m), 1603 (w), 1585 (w), 1493 (w), 1451 (w), 1382 (w), 1316 (w), 1273 (s), 1227 (m), 1175 (w), 1152 (w), 1112 (m), 1069 (m), 1046 (m), 1027 (m), 954 (m), 887 (w), 856 (w), 822 (w), 757 (w), 710 (s), 686 (m), 663 (w), 637 (w), 600 (w), 559 (m), 524 (w).

HR-MS: (ESI, 70 eV)=m/z calculated for: C₃₄H₄₉O₅[M+H]⁺ 537.35745 u, C₃₄H₄₈O₅Na [M+Na]⁺ 559.33940 u, found: 537.35769 u, 559.33938.

[α]_{λ}²⁰: c=0.56 g/100 mL, CHCl₃:
[α]₃₆₅²⁰=201.62° (±0.29°),
[α]₄₃₆²⁰=111.59° (±0.22°),
[α]₅₄₆²⁰=63.60° (±0.44°),
[α]₅₇₉²⁰=55.44° (±0.34°),
[α]₅₈₉²⁰=52.73° (±0.37°),

### Synthesis of 4-(7β,19-epoxy-5α-hydroxy-6-on-cholestan-3β-yl)-1-methyl ester of itaconic acid (21) [WIL-367]

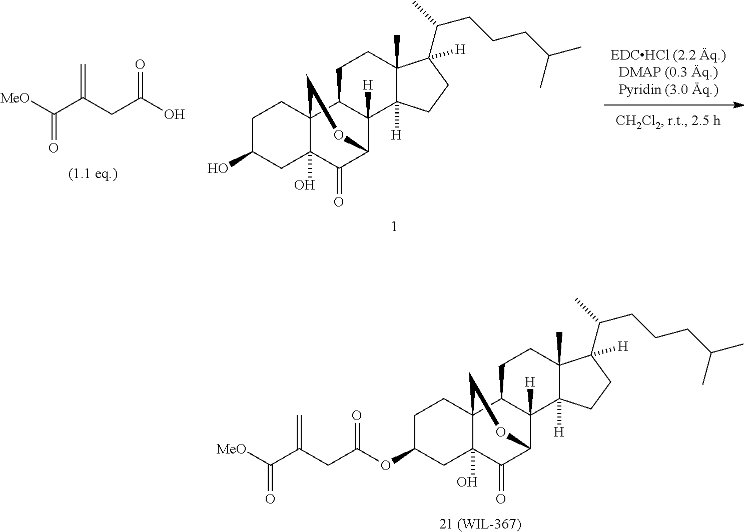

To a solution of 73 mg (0.169 mmol, 1.0 eq.) of alcohol 1 in 1.7 mL of dichloromethane were added 0.042 ml (0.54 mmol, 3.2 eq.) of pyridine, 74 mg (0.39 mmol, 2.3 eq.) of EDC·HCl, 8 mg (0.07 mmol, 0.4 eq.) of DMAP and 28 mg (0.19 mmol, 1.1 eq.) of monomethyl ester of itaconic acid under an argon atmosphere. The reaction mixture was stirred at room temperature for 2.5 h. After the reaction was complete (TLC), the reaction mixture was diluted with 10 ml of ethyl acetate. The organic phase was washed once each with saturated aqueous NaHCO₃ solution, water and saturated aqueous NaCl solution. The organic phase was subsequently dried over MgSO₄, and the solvent was removed under reduced pressure. The residue was purified by column chromatography on silica (10:1-8:1; cHex/EtOAc), and the product 21 could be obtained in the form of a colorless solid (65 mg, 0.116 mmol, 69%).

Analytics relating to 21:

Melting point: 132° C.-134° C.

Rf: 0.39 (3:1, cHex:EtOAc).

¹H NMR: (500 MHz, CDCl₃) δ [ppm]: 6.31 (d, J=1.0 Hz, 1H), 5.69 (d, J=1.1 Hz, 1H), 5.25 (tt, J=11.1, 5.4 Hz, 1H), 4.13 (d, J=10.5 Hz, 1H), 3.90 (d, J=10.2 Hz, 1H), 3.81 (d, J=1.7 Hz, 1H), 3.76 (s, 3H), 3.30 (d, J=1.1 Hz, 2H), 2.57 (s, 1H), 2.12-2.02 (m, 3H), 2.00-1.94 (m, 2H), 1.91-1.82 (m, 1H), 1.72 (td, J=13.8, 4.7 Hz, 1H), 1.63-1.47 (m, 4H), 1.42-1.06 (m, 14H), 1.02-0.95 (m, 1H), 0.89 (d, J=6.5 Hz, 3H), 0.86 (dd, J=6.6, 2.4 Hz, 6H), 0.74 (s, 3H).

¹³C NMR: (126 MHz, CDCl₃) δ [ppm]: 212.83, 170.14, 166.77, 133.91, 128.55, 78.65, 76.16, 69.79, 63.54, 55.73, 52.27, 52.02, 45.52, 45.09, 43.22, 40.53, 40.24, 39.59, 38.19, 36.22, 35.77, 35.14, 28.64, 28.14, 26.10, 23.89, 22.94, 22.89, 22.69, 21.70, 21.24, 18.74, 13.07.

FT-IR (ATR): ν [cm⁻¹]=3422 (bw), 2951 (w), 2930 (w), 2866 (w), 1731 (s), 1644 (w), 1457 (w), 1434 (w), 1375 (w), 1333 (m), 1301 (w), 1201 (s), 1178 (s), 1146 (m), 1097 (w), 1040 (m), 1033 (m), 1023 (m), 975 (w), 956 (m), 936 (w), 920 (w), 884 (w), 826 (m), 813 (m), 783 (w), 754 (m), 712 (w), 644 (w), 584 (w), 562 (m), 545 (w), 459 (w).

HR-MS: (ESI, 70 eV)=m/z calculated for: C₃₄H₅₀O₇[M+H]⁺ 559.36293 u, C₃₃H₅₀O₇Na [M+Na]⁺ 581.34488 u, found: 559.36329 u, 581.34505 u.

[α]_{λ}²⁰: c=0.52 g/100 mL, CHCl₃:
[α]₃₆₅²⁰=75.000,
[α]₄₃₆²⁰=64.81°,
[α]₅₄₆²⁰=38.14°,
[α]₅₇₉²⁰=33.72°,
[α]₅₈₉²⁰=33.79°,

### Synthesis of 7β,19-epoxy-3β,5α-hydroxy-cholestan-6-on-oxime (22) [WIL-368]

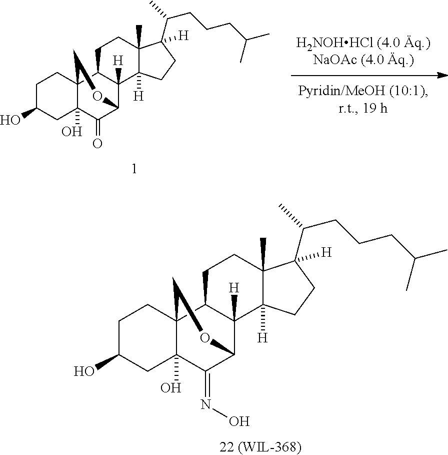

To a solution of 101 mg (0.231 mmol, 1.0 eq.) of alcohol 1 in 2.3 mL of pyridine were added 64 mg (0.93 mmol, 4.0 eq.) of hydroxylamine hydrochloride and a solution of 78 mg (0.93 mmol, 4.0 eq.) of sodium acetate in 0.5 ml of methanol at room temperature. The reaction mixture was stirred at room temperature for 19 h, and after the reaction was complete (TLC), the reaction mixture was diluted with EtOAc and a saturated aqueous NaCl solution. The aqueous phase was washed with a diluted NaCl solution, water, a saturated aqueous NaCl solution, and a CuSO₄ solution. The organic phase was dried over MgSO₄, and the solvent was removed under reduced pressure. The residue was recrystallized from a mixture of methanol, ethyl acetate and water. The product 22 could be obtained as a colorless solid (35 mg, 0.078 mmol, 34%).

Analytics relating to 22:

Melting point: 266° C.-267° C. (decomposition).

Rf: 0.09 (1:1, cHex:EtOAc); 0.45 (5:5:1, cHex:EtOAc:MeOH)

¹H NMR: (600 MHz, DMSO-d₆) δ [ppm]: 11.02 (s, 1H), 4.62 (s, 1H), 4.36 (d, J=5.9 Hz, 1H), 3.84 (d, J=9.5 Hz, 2H), 3.69 (s, 1H), 3.59 (d, J=9.9 Hz, 1H), 1.95 (d, J=12.7 Hz, 1H), 1.82-1.65 (m, 3H), 1.65-1.57 (m, 1H), 1.55-1.43 (m, 3H), 1.42-1.17 (m, 8H), 1.17-0.99 (m, 8H), 0.97-0.90 (m, 1H), 0.85 (d, J=6.5 Hz, 3H), 0.81 (dd, J=6.6, 2.9 Hz, 6H), 0.67 (s, 3H).

¹³C NMR: (151 MHz, DMSO-d₆) δ [ppm]: 158.76, 74.65, 71.50, 63.77, 62.83, 55.16, 52.23, 44.83, 44.31, 42.47, 40.22, 38.92, 38.78, 37.95, 35.67, 35.11, 29.88, 28.23, 27.39, 23.15, 22.67, 22.44, 22.41, 21.13, 21.11, 18.47, 12.78.

FT-IR (ATR): ν [cm¹]=3511 (bw), 3290 (bw), 3197 (bw), 3111 (bw), 2954 (m), 2939 (m), 2921 (m), 2867 (m), 1493 (w), 1469 (m), 1444 (w), 1418 (w), 1384 (w), 1375 (w), 1362 (w), 1337 (w), 1307 (w), 1251 (w), 1208 (w), 1181 (w), 1157 (m), 1131 (w), 1098 (w), 1069 (w), 1042 (m), 1018 (m), 986 (m), 944 (s), 906 (m), 894 (w), 873 (w), 838 (w), 811 (m), 789 (m), 733 (m), 712 (w), 666 (w), 605 (w), 593 (w), 578 (w), 562 (m), 488 (w), 462 (w), 443 (w).

HR-MS: (ESI, 70 eV)=m/z calculated for: C₂₇H₄₆NO₄ [M+H]⁺ 448.34214 u, found: 448.34203 u.

[α]_{λ}²⁰: c=0.59 g/100 mL, CHCl₃:
[α]₃₆₅²⁰=141.08° (±0.16°),
[α]₄₃₆²⁰=84.58° (±0.00°),
[α]₅₄₆²⁰=48.41° (±0.08°),
[α]₅₇₉²⁰=41.58° (±0.16°),
[α]₅₈₉²⁰=38.03° (±0.16°),

### Synthesis of (7β,19-epoxy-5α-hydroxy-6-on-cholestan-3β-yl)methyl ester of fumaric acid (23) [WIL-369]

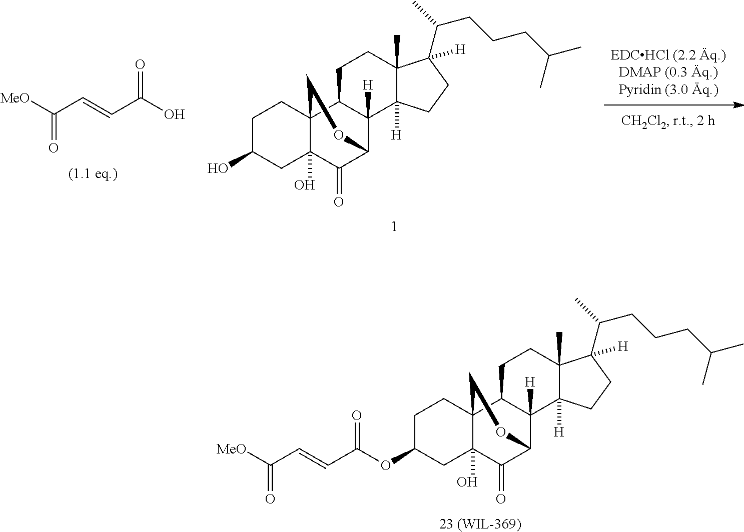

To a solution of 100 mg (0.23 mmol, 1.0 eq.) of alcohol 1 in 2.3 mL of dichloromethane were added 0.06 ml (0.69 mmol, 3.0 eq.) of pyridine, 97 mg (0.051 mmol, 2.2 eq.) of EDC·HCl, 8 mg (0.07 mmol, 0.3 eq.) of DMAP and 37 mg (0.25 mmol, 1.1 eq.) of monomethyl ester of fumaric acid under an argon atmosphere. The reaction mixture was stirred at room temperature for 2 h. After the reaction was complete (TLC), the reaction mixture was diluted with 20 ml of ethyl acetate. The organic phase was washed once each with saturated aqueous NaHCO₃ solution, water and saturated aqueous NaCl solution. The organic phase was subsequently dried over MgSO₄, and the solvent was removed under reduced pressure. The residue was purified by column chromatography on silica (8:1-6:1; cHex/EtOAc), and the product 23 could be obtained in the form of a colorless solid (106 mg, 0.195 mmol, 84%).

Analytics relating to 23:

Melting point: 153° C.-154° C.

Rf: 0.20 (6:1, cHex:EtOAc).

¹H NMR: (500 MHz, CDCl₃) δ [ppm]: 6.85 (d, J=1.1 Hz, 2H), 5.36 (tt, J=11.1, 5.5 Hz, 1H), 4.17 (dd, J=10.3, 1.7 Hz, 1H), 3.94 (d, J=10.3 Hz, 1H), 3.85 (d, J=1.7 Hz, 1H), 3.83 (s, 3H), 2.67 (s, 1H), 2.18-2.01 (m, 5H), 1.94-1.84 (m, 1H), 1.77 (td, J=13.8, 4.6 Hz, 1H), 1.69-1.57 (m, 3H), 1.53 (dt, J=13.1, 6.6 Hz, 1H), 1.45-1.08 (m, 14H), 1.04-0.97 (m, 1H), 0.91 (d, J=6.5 Hz, 3H), 0.88 (dd, J=6.6, 2.4 Hz, 6H), 0.76 (s, 3H).

¹³C NMR: (126 MHz, CDCl₃) δ [ppm]: 212.69, 165.54, 164.36, 134.09, 133.44, 78.61, 76.15, 70.51, 63.52, 55.73, 52.46, 52.01, 45.52, 45.08, 43.22, 40.54, 40.23, 39.59, 36.22, 35.76, 35.16, 28.64, 28.13, 26.12, 23.88, 22.93, 22.89, 22.69, 21.70, 21.23, 18.74, 13.07.

FT-IR (ATR): ν [cm⁻¹]=3453 (bw), 2951 (m), 2868 (m), 1722 (s), 1645 (w), 1495 (w), 1437 (w), 1383 (w), 1302 (s), 1260 (m), 1227 (w), 1155 (s), 1100 (w), 1086 (w), 1033 (m), 1021 (m), 977 (m), 910 (w), 889 (w), 825 (w), 775 (w), 757 (w), 734 (w), 676 (w), 593 (w), 559 (w), 529 (w).

HR-MS: (ESI, 70 eV)=m/z calculated for: C₃₃H₄₈O₇[M+H]⁺ 545.34728 u, C₃₂H₄₈O₇Na [M+Na]⁺ 567.32922 u, found: 545.34756 u, 567.32935 u.

[α]_{λ}²⁰: c=0.59 g/100 mL, CHCl₃:
[α]₃₆₅²⁰=141.08° (±0.16°),
[α]₄₃₆²⁰=84.58° (±0.00°),
[α]₅₄₆²⁰=48.41° (±0.08°),
[α]₅₇₉²⁰=41.58° (±0.16°),
[α]₅₈₉²⁰=38.03° (±0.16°),

### Synthesis of (7β,19-epoxy-5α-hydroxy-6-on-cholestan-3β-yl) ester of methanesulfonic acid (24) [TAS-S389]

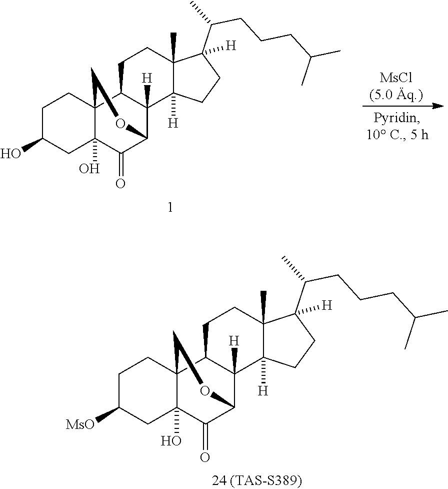

To a solution of 150 mg (0.347 mmol, 1.0 eq.) of alcohol 1 in 8 mL of pyridine was added 138 ml (1.75 mmol, 5.0 eq.) of mesyl chloride, followed by stirring at 10° C. for 5 h. Subsequently, the reaction mixture was quenched by adding water, and the aqueous phase was extracted with ethyl acetate. The combined organic phases were successively washed with water, 1 N HCl, and a saturated aqueous NaCl solution. The organic phase was subsequently dried over MgSO₄, and the solvent was removed under reduced pressure. The residue was purified by column chromatography on silica (2:1, cHex/EtOAc), and the mesitylated product 24 could be obtained in the form of a colorless solid (175 mg, 0.343 mmol, 99%).

Analytics relating to 24:

Melting point: 173° C.-176° C.

Rf: 0.35 (2:1, cHex:EtOAc).

¹H NMR: (600 MHz, CDCl₃) δ [ppm]: 5.12 (dp, J=11.0, 5.7 Hz, 1H), 4.14-4.08 (m, 1H), 3.92 (d, J=10.3 Hz, 1H), 3.84 (d, J=1.7 Hz, 1H), 3.01 (s, 3H), 2.77 (s, 1H), 2.24 (ddd, J=13.0, 5.4, 1.6 Hz, 1H), 2.21-2.07 (m, 3H), 2.09-2.03 (m, 1H), 1.91-1.82 (m, 1H), 1.81-1.70 (m, 2H), 1.62-1.47 (m, 3H), 1.42-1.05 (m, 14H), 1.02-0.95 (m, 1H), 0.89 (d, J=6.5 Hz, 3H), 0.86 (dd, J=6.6, 2.9 Hz, 6H), 0.75-0.73 (m, 3H).

¹³C NMR: (151 MHz, CDCl₃) δ [ppm]: 212.21, 78.67, 77.47, 76.06, 63.35, 55.71, 51.95, 45.49, 45.03, 43.13, 40.35, 40.18, 39.57, 38.70, 36.50, 36.19, 35.73, 28.61, 28.11, 27.56, 23.86, 22.92, 22.85, 22.67, 21.67, 21.36, 18.72, 13.05.

FT-IR ν [cm⁻¹]=3499 (w), 2947 (w), 2921 (w), 2865 (w), 1733 (m), (ATR): 1496 (w), 1467 (w), 1455 (w), 1417 (w), 1363 (m), 1356 (m), 1334 (m), 1302 (m), 1262 (w), 1229 (w), 1181 (m), 1169 (s), 1151 (m), 1100 (w), 1087 (w), 1045 (m), 1019 (w), 981 (m), 945 (s), 885 (m), 861 (s), 821 (m), 764 (m), 748 (m), 720 (w), 662 (w), 638 (w), 592 (w), 559 (w), 531 (m), 507 (m), 497 (m), 434 (w).

HR-MS: (ESI, 70 eV)=m/z calculated for: C₂₈H₄₆O₆SNa [M+Na]⁺533.290731 u, found: 533.29086 u.

[α]₃₆₅²⁰: c=0.54 g/100 mL, CHCl₃:
[α]₃₆₅²⁰=131.61° (±0.23°),
[α]₄₃₆²⁰=75.81° (±0.08°),
[α]₅₄₆²⁰=43.52° (±0.09°),
[α]₅₇₉²⁰=38.12° (±0.15°),
[α]₅₈₉²⁰=36.28° (±0.00°),

### Synthesis of 7β,19-epoxy-5α-hydroxy-cholest-2-en-6-one (25) [TAS-S392]

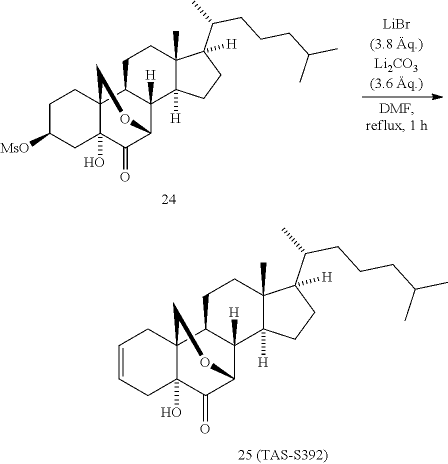

To a solution of 143 mg (0.280 mmol, 1.0 eq.) of mesylate in 5.0 mL of DMF were added 92 mg (1.06 mmol, 3.8 eq.) of LiBr and 99 mg (1.01 mmol, 3.6 eq.) of Li₂CO₃. The reaction mixture was subsequently heated under reflux for 1 h. After the reaction mixture had cooled down to room temperature, the solvent was removed under reduced pressure, the residue was taken up in water and dichloromethane, and the aqueous phase was extracted with DCM. The combined organic phases were dried with MgSO₄, and the solvent was removed under reduced pressure. The residue was purified by column chromatography on silica (15:1, cHex/EtOAc), and the elimination product 25 could be obtained in the form of a colorless solid (75 mg, 0.18 mmol, 65%).

Analytics relating to 25:

Melting point: 78° C.-80° C.

Rf: 0.56 (2:1, cHex:EtOAc).

¹H NMR: (600 MHz, CDCl₃) δ [ppm]: 5.76-5.66 (m, 2H), 3.91-3.80 (m, 3H), 2.76-2.64 (m, 2H), 2.19-2.08 (m, 3H), 2.06 (dt, J=13.1, 3.1 Hz, 1H), 1.87 (dtd, J=13.1, 9.4, 5.9 Hz, 1H), 1.72-1.36 (m, 8H), 1.35-1.05 (m, 9H), 1.02-0.95 (m, 1H), 0.89 (d, J=6.5 Hz, 3H), 0.85 (dd, J=6.6, 3.0 Hz, 6H), 0.75 (s, 3H).

¹³C NMR: (151 MHz, CDCl₃) δ [ppm]: δ 214.86, 125.53, 123.65, 77.16, 77.08, 65.09, 55.74, 51.94, 45.48, 45.35, 43.58, 40.36, 40.26, 39.59, 36.21, 35.77, 33.46, 28.65, 28.12, 24.66, 23.88, 22.93, 22.86, 22.68, 21.54, 18.74, 13.09.

FT-IR ν [cm⁻¹]=3568 (bw), 3464 (bw), 3399 (bw), 3023 (w), 2949 (s), (ATR): 2933 (m), 1895 (m), 2865 (m), 1742 (s), 1646 (w), 1615 (w), 1490 (w), 1464 (m), 1425 (m), 1380 (m), 1366 (w), 1336 (w), 1314 (w), 1285 (w), 1257 (w), 1232 (w), 1207 (w), 1178 (m), 1128 (w), 1099 (w), 1077 (m), 1039 (s), 979 (w), 966 (w), 932 (w), 908 (m), 899 (m), 876 (w), 861 (w), 820 (w), 801 (w), 770 (w), 738 (m), 657 (s), 608 (m), 565 (m), 488 (w), 463 (w), 429 (w).

HR-MS: (ESI, 70 eV)=m/z calculated for: C₂₇H₄₃O₃[M+H]⁺ 415.320671 u, C₂₇H₄₂O₃Na [M+Na]⁺ 437.302616 u, found: 415.32086 u, 437.30267 u.

[α]_{λ}²⁰: c=0.55 g/100 mL, CHCl₃:
[α]₃₆₅²⁰=663.93° (±0.31°),
[α]₄₃₆²⁰=290.97° (±0.08°),
[α]₅₄₆²⁰=146.73° (±0.15°),
[α]₅₇₉²⁰=125.88° (±0.08°),
[α]₅₈₉²⁰=119.94° (±0.22°),

### Synthesis of 7β,19-epoxy-5α-hydroxy-cholestan-6-one (26) [TAS-S398]

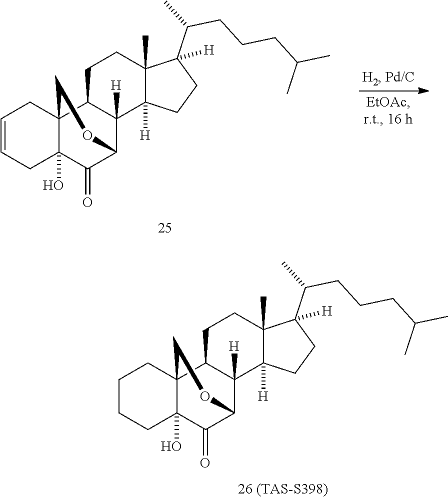

To a solution of 70 mg (0.17 mmol, 1.0 eq.) of alkene 25 in 4 ml of ethyl acetate was added 20 mg of Pd/C, and the reaction mixture was stirred at room temperature under a hydrogen atmosphere for 16 h. Subsequently, the reaction mixture was filtered through celite, and the solvent was removed under reduced pressure. The residue was purified by column chromatography on silica (5:1, cHex/EtOAc), and the hydrogenation product 26 could be obtained in the form of a colorless solid (60 mg, 0.14 mmol, 85%). Analytics relating to 26:

Melting point: 50° C.

Rf: 0.58 (3:1, cHex:EtOAc).

¹H NMR: (500 MHz, CDCl₃) δ [ppm]: 4.15 (dd, J=10.1, 1.6 Hz, 1H), 3.86 (d, J=10.1 Hz, 1H), 3.79 (d, J=1.7 Hz, 1H), 2.43-2.36 (m, 1H), 2.07 (tdd, J=11.0, 10.1, 9.5, 2.4 Hz, 2H), 1.94 (dd, J=13.8, 4.5 Hz, 1H), 1.92-1.75 (m, 2H), 1.64-1.55 (m, 6H), 1.51 (ddt, J=16.5, 6.6, 3.2 Hz, 2H), 1.44-1.06 (m, 14H), 1.03-0.95 (m, 1H), 0.89 (d, J=6.5 Hz, 3H), 0.86 (dd, J=6.6, 2.5 Hz, 6H), 0.74 (s, 3H).

¹³C NMR: (126 MHz, CDCl₃) δ [ppm]: 215.48, 77.89, 76.46, 63.78, 55.75, 52.13, 45.57, 45.05, 44.07, 40.83, 40.33, 39.60, 36.23, 35.78, 30.11, 28.67, 28.13, 23.88, 22.94, 22.91, 22.69, 22.18, 21.43, 20.32, 19.14, 18.75, 13.08.

FT-IR ν [cm⁻¹]=3474 (bw), 2932 (s), 2866 (s), 1734 (s), 1492 (w), (ATR): 1465 (m), 1449 (m), 1382 (m), 1346 (w), 1261 (w), 1237 (w), 1205 (w), 1153 (m), 1132 (w), 1103 (w), 1085 (w), 1043 (s), 1006 (m), 988 (m), 962 (w), 946 (w), 906 (w), 879 (m), 825 (w), 804 (w), 749 (s), 689 (w), 660 (w), 632 (w), 558 (s), 538 (w), 473 (w), 463 (w), 453 (w).

HR-MS: (ESI, 70 eV)=m/z calculated for: C₂₇H₄₅O₃[M+H]⁺ 417.336322 u, C₂₇H₄₄O₃Na [M+Na]⁺ 439.318266 u, found: 417.33657 u, 439.31838 u.

[α]_{λ}²⁰: c=0.49 g/100 mL, CHCl₃:
[α]₃₆₅²⁰=338.16° (±0.16°),
[α]₄₃₆²⁰=153.74° (±0.10°),
[α]₅₄₆²⁰=80.07° (±0.09°),
[α]₅₇₉²⁰=69.18° (±0.17°),
[α]₅₅₉²⁰=65.78° (±0.10°),

### Synthesis of 7β,19-epoxy-6-methylen-cholest-3β,5α-diol (27) [TAS-S399]

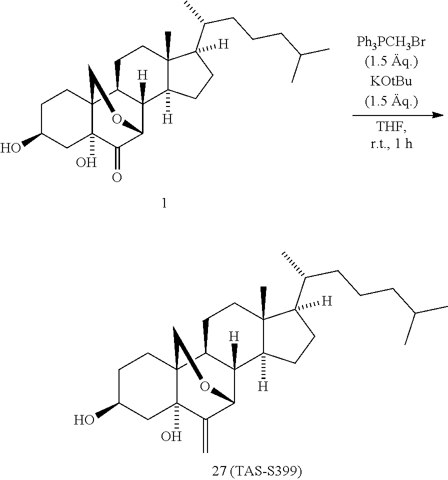

To a solution of 125 mg (0.35 mmol, 1.5 eq.) of Ph₃PCH₃Br in 2 mL of dry THF was added 39 mg (0.35 mmol, 1.5 eq.) of KO^{t}Bu at 0° C. The reaction mixture was warmed up to room temperature, stirred at room temperature for 30 min, and subsequently cooled down again to 0° C. Subsequently, a solution of 100 mg (0.231 mmol, 1.0 eq.) of alcohol 1 in 3.0 ml of THF was added to the reaction mixture, and subsequently warmed up to room temperature. The reaction was stopped after h by adding water. The aqueous phase was extracted with ethyl acetate, and the combined organic phases were washed with saturated aqueous NaCl solution, dried over MgSO₄, and freed from solvent under reduced pressure. The residue was purified by column chromatography on silica (1:1, cHex/EtOAc), and the alkene 27 could be obtained in the form of a colorless solid (82 mg, 0.19 mmol, 83%).

Analytics relating to 27:

Melting point: 171° C.-174° C.

Rf: 0.20 (1:2, cHex:EtOAc).

¹H NMR: (500 MHz, CDCl₃) δ [ppm]: 5.23 (s, 1H), 4.97 (s, 1H), 4.10 (tt, J=11.0, 5.2 Hz, 1H), 3.98 (dd, J=10.0, 1.7 Hz, 1H), 3.91 (s, 1H), 3.71 (d, J=9.9 Hz, 1H), 2.11-2.03 (m, 2H), 1.93-1.80 (m, 4H), 1.69-1.61 (m, 3H), 1.54-1.47 (m, 2H), 1.45-1.06 (m, 16H), 1.02-0.95 (m, 1H), 0.90 (d, J=6.5 Hz, 3H), 0.86 (dd, J=6.6, 2.1 Hz, 6H), 0.75 (s, 3H).

¹³C NMR: (126 MHz, CDCl₃) δ [ppm]: 155.41, 109.49, 75.66, 75.24, 66.82, 63.83, 55.86, 53.00, 45.44, 44.94, 43.90, 43.40, 40.82, 39.62, 39.12, 36.29, 35.76, 30.24, 28.79, 28.15, 23.86, 22.96, 22.94, 22.70, 21.88, 21.71, 18.77, 13.37.

FT-IR ν [cm⁻¹]=3436 (bw), 1934 (s), 1906 (s), 2865 (s), 1657 (w), (ATR): 1489 (w), 1462 (m), 1450 (m), 1437 (m), 1415 (w), 1382 (m), 1365 (m), 1310 (m), 1263 (m), 1222 (m), 1171 (w), 1151 (w), 1122 (w), 1073 (s), 1039 (s), 1029 (s), 1013 (s), 1001 (m), 956 (s), 926 (m), 892 (m), 875 (m), 852 (m), 818 (m), 767 (w), 749 (w), 710 (w), 693 (m), 660 (w), 531 (m), 491 (m), 454 (m).

[α]_{λ}²⁰: c=0.57 g/100 mL, CHCl₃:
[α]₃₆₆²⁰=85.67° (±0.30°),
[α]₄₃₆²⁰=56.78° (±0.08°),
[α]₅₄₆²⁰=34.45° (±0.17°),
[α]₅₇₉²⁰=30.41° (±0.17°),
[α]₅₈₉²⁰=29.12° (±0.00°),

### Synthesis of 3α,5α-dihydroxy-7β,19-epoxy-cholestan-6-one (28) [TAS-S403]

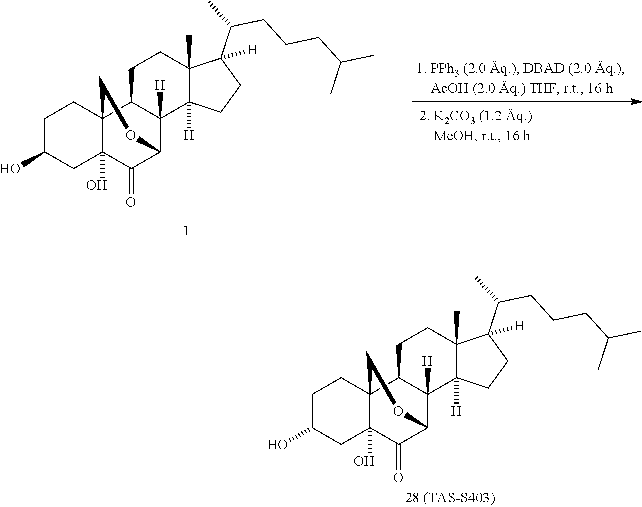

To a solution of 121 mg (0.46 mmol, 2.0 eq.) of PPh₃ and 106 mg (0.46 mmol, 2.0 eq.) of DBAD in 2.0 ml of dry THF was added a solution of 100 mg (0.23 mmol, 1.0 eq.) of alcohol 1 in 3.0 ml of dry THF. When the color of the reaction mixture turned from colorless to yellow, 26 ml (0.46 mmol, 2.0 eq.) of acetic acid was added, followed by stirring at room temperature for 16 h. Subsequently, the reaction mixture was adsorbed on silica, and the solvent was removed under reduced pressure. The residue was purified by column chromatography on silica (20:1-6:1; cHex/EtOAc), and the acetate obtained (0.46 mmol, 2.0 eq.) was directly dissolved in 5.0 ml of methanol. To this solution, 34 mg (0.25 mmol, 1.2 eq.) of K₂CO₃ was added, the reaction mixture was stirred at room temperature for 16 h, and subsequently quenched by adding water. The aqueous phase was extracted with ethyl acetate, the combined organic phases were washed with saturated aqueous NaCl solution, dried over MgSO₄, and the solvent was removed under reduced pressure. The residue was purified by column chromatography on silica (2:1, cHex/EtOAc), and the free alcohol 28 could be obtained in the form of a colorless solid (80 mg, 0.18 mmol, 80%).

Analytics relating to 28:

Melting point: 148° C.-150° C.

Rf: 0.11 (2:1, cHex:EtOAc).

¹H NMR: (500 MHz, CDCl₃) δ [ppm]: 4.18 (p, J=2.9 Hz, 1H), 3.97 (dd, J=10.3, 1.6 Hz, 1H), 3.92 (d, J=10.2 Hz, 1H), 3.81 (d, J=1.6 Hz, 1H), 3.62 (bs, 2H), 2.13-2.04 (m, 4H), 1.92-1.76 (m, 4H), 1.69-1.55 (m, 2H), 1.51 (dt, J=13.1, 6.6 Hz, 1H), 1.47-1.07 (m, 14H), 1.03-0.94 (m, 1H), 0.90 (d, J=6.5 Hz, 3H), 0.86 (dd, J=6.6, 2.5 Hz, 6H), 0.74 (s, 3H).

¹³C NMR: (126 MHz, CDCl₃) δ [ppm]: 212.36, 79.55, 76.56, 66.47, 63.64, 55.73, 51.99, 45.55, 45.08, 43.63, 41.35, 40.24, 39.58, 36.21, 35.77, 35.01, 28.71, 28.63, 28.12, 23.89, 22.93, 22.87, 22.68, 21.46, 18.74, 17.62, 13.06.

FT-IR ν [cm⁻¹]=3428 (bw), 3287 (bw), 2946 (s), 2867 (m), 1735 (s), (ATR): 1459 (m), 1439 (m), 1377 (m), 1344 (w), 1327 (w), 1297 (w), 1244 (m), 1211 (m), 1119 (m), 1089 (m), 1079 (m), 1044 (s), 949 (m), 934 (m), 921 (m), 897 (m), 860 (m), 821 (w), 766 (w), 730 (w), 714 (w), 667 (m), 632 (m), 597 (m), 581 (m), 526 (w), 492 (w), 453 (w).

HR-MS: (ESI, 70 eV)=m/z calculated for: C₂₇H₄₅O₄[M+H]⁺ 433.331236 u, C₂₇H₄₄O₄Na [M+Na]⁺ 455.313181 u, found: 433.33145 u, 455.31325 u.

[α]_{λ}²⁰: c=0.53 g/100 mL, CHCl₃:
[α]₃₆₅²⁰=181.70° (±0.71°),
[α]₄₃₆²⁰=82.45° (±0.53°),
[α]₅₄₆²⁰=42.89° (±0.32°),
[α]₅₇₉²⁰=36.22° (±0.26°),
[α]₅₈₉²⁰=32.20° (±0.32°),

### Synthesis of 3α-acetoxy-5α-hydroxy-7β,19-epoxy-cholestan-6-one (29) [TAS-S405]

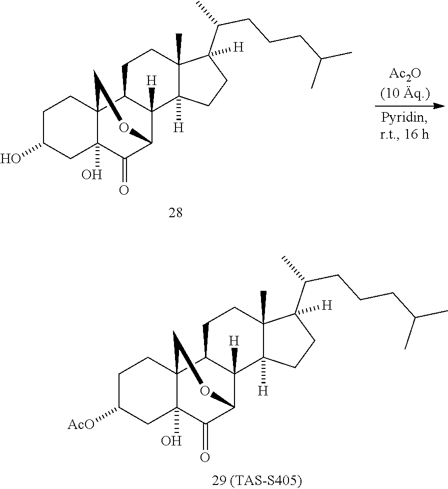

To a solution of 50 mg (0.116 mmol, 1.0 eq.) of alkohol 28 in 2.0 ml of pyridine was added 110 ml (1.16 mmol, 10 eq.) of Ac₂O. The reaction mixture was stirred at room temperature for 16 h, and then diluted with water. The aqueous phase was extracted with ethyl acetate, the combined organic phases were washed with 1 M HCl and saturated aqueous NaCl solution, and dried over MgSO₄. The solvent was removed under reduced pressure, and the residue was purified by column chromatography on silica (2:1, cHex/EtOAc). The acetate 29 could be obtained in the form of a colorless solid (54 mg, 0.114 mmol, 98%).

Analytics relating to 29:

Melting point: 122° C.-114° C.

Rf: 0.43 (2:1, cHex:EtOAc).

¹H NMR: (500 MHz, CDCl₃) δ [ppm]: 5.31-5.26 (m, 1H), 3.94 (s, 2H), 3.77 (d, J=1.6 Hz, 1H), 3.33 (s, 1H), 2.23-2.11 (m, 2H), 2.13-2.03 (m, 5H), 1.92-1.75 (m, 5H), 1.66-1.56 (m, 1H), 1.51 (dt, J=13.1, 6.6 Hz, 1H), 1.46-1.23 (m, 9H), 1.22-1.05 (m, 5H), 1.03-0.95 (m, 1H), 0.90 (d, J=6.5 Hz, 3H), 0.86 (dd, J=6.6, 2.5 Hz, 6H), 0.75 (s, 3H).

¹³C NMR: (126 MHz, CDCl₃) δ [ppm]: 210.28, 169.44, 77.16, 76.67, 68.81, 63.38, 55.74, 52.04, 45.57, 44.58, 43.32, 41.07, 40.25, 39.59, 36.22, 35.80, 33.15, 28.67, 28.13, 25.47, 23.92, 22.94, 22.89, 22.69, 21.58, 21.53, 18.75, 18.10, 13.05.

FT-IR ν [cm⁻¹]=3484 (bw), 2948 (m), 2932 (m), 2866 (m), 1737 (s), (ATR): 1465 (w), 1441 (w), 1375 (m), 1248 (m), 1224 (m), 1157 (m), 1119 (w), 1094 (m), 1083 (m), 1043 (s), 969 (w), 914 (w), 893 (w), 852 (w), 832 (m), 769 (w), 749 (m), 713 (w), 657 (w), 631 (w), 606 (w), 563 (m), 524 (w), 494 (w), 427 (w).

HR-MS: (ESI, 70 eV)=m/z calculated for: C₂₉H₄₇O₅[M+H]⁺ 475.241801 u, C₂₉H₄₆O₅Na [M+Na]⁺ 497.323746 u, found: 475.34182 u, 497.32366 u.

[α]_{λ}²⁰: c=0.55 g/100 mL, CHCl₃:
[α]₃₆₅²⁰=87.15° (±0.17°),
[α]₄₃₆²⁰=52.85° (±0.08°),
[α]₅₄₆²⁰=30.97° (±0.22°),
[α]₅₇₉²⁰=27.76° (±0.31°),
[α]₅₈₉²⁰=26.12° (±0.17°),

### Synthesis of 7β,19-epoxy-6α-methylcholestan-3β,5α,6β-triol (30) [TAS-S406]

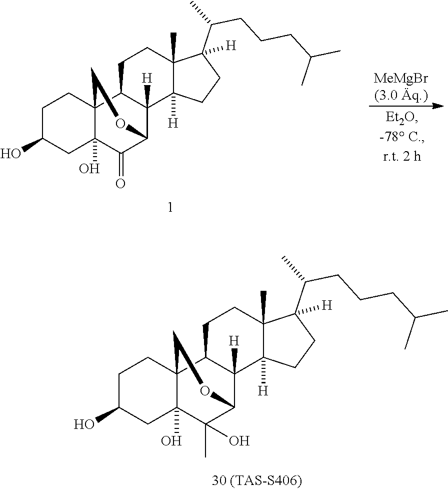

To a solution of 100 mg (0.23 mmol, 1.0 eq.) of ketone 1 in 5.0 mL of dry diethyl ether was added 0.23 ml (0.69 mmol, 3.0 eq., 3.0 M in Et₂O) of MeMgBr at −78° C. The reaction mixture was slowly warmed up to room temperature, and stirred at room temperature for 2 h. The complete conversion of the starting material was monitored by means of TLC controls, and subsequently, the reaction was stopped by adding a saturated NH₄Cl solution. The aqueous phase was extracted with ethyl acetate, the combined organic phases were washed with saturated aqueous NaCl solution, dried over MgSO₄, and the solvent was removed under reduced pressure. The residue was purified by column chromatography on silica (1:5, cHex/EtOAc), and the alcohol 30 could be obtained in the form of a colorless solid (90 mg, 0.20 mmol, 87%).

Analytics relating to 30:

Melting point: 218° C.-221° C.

Rf: 0.16 (1:5, cHex:EtOAc).

¹H NMR: (500 MHz, CDCl₃) δ [ppm]: 4.04 (tt, J=10.9, 5.1 Hz, 1H), 3.87 (dd, J=10.0, 1.6 Hz, 1H), 3.63 (d, J=10.0 Hz, 1H), 3.34 (d, J=1.2 Hz, 1H), 2.49 (s, 3H), 2.07-1.98 (m, 2H), 1.90-1.75 (m, 3H), 1.69-1.46 (m, 6H), 1.43-1.23 (m, 11H), 1.21-1.07 (m, 6H), 1.03-0.96 (m, 1H), 0.90 (d, J=6.5 Hz, 3H), 0.86 (dd, J=6.6, 2.3 Hz, 6H), 0.71 (s, 3H).

¹³C NMR: (126 MHz, CDCl₃) δ [ppm]: 76.81, 74.91, 67.92, 64.05, 55.91, 54.37, 45.39, 42.98, 40.80, 40.68, 40.00, 39.64, 39.30, 36.32, 35.85, 30.20, 28.80, 28.15, 24.09, 23.91, 22.95, 22.91, 22.70, 22.63, 22.07, 18.77, 12.69.

FT-IR ν [cm⁻¹]=3449 (bw), 2930 (m), 2847 (m), 1492 (w), 1463 (w), (ATR): 1438 (w), 1378 (m), 1365 (w), 1325 (w), 1279 (w), 1226 (w), 1171 (w), 1125 (w), 1107 (m), 1067 (m), 1043 (s), 982 (w), 960 (w), 935 (m), 920 (w), 881 (w), 852 (w), 819 (w), 757 (w), 646 (m), 523 (m).

HR-MS: (ESI, 70 eV)=m/z calculated for: C₂₈H₄₈O₄Na [M+Na]⁺471.344481 u, found: 471.34455 u.

[α]_{λ}²⁰: c=0.54 g/100 mL, CHCl₃:
[α]₃₆₅²⁰=252.10° (±0.17°),
[α]₄₃₆²⁰=157.41° (±0.79°),
[α]₅₄₆²⁰=92.28° (±0.61°),
[α]₅₇₉²⁰=81.05° (±0.08°),
[α]₅₈₉²⁰=77.10° (±0.08°),

### Synthesis of 5α-hydroxy-2α,3α-7β,19-diepoxy-cholestan-6-one (31) [TAS-S407]

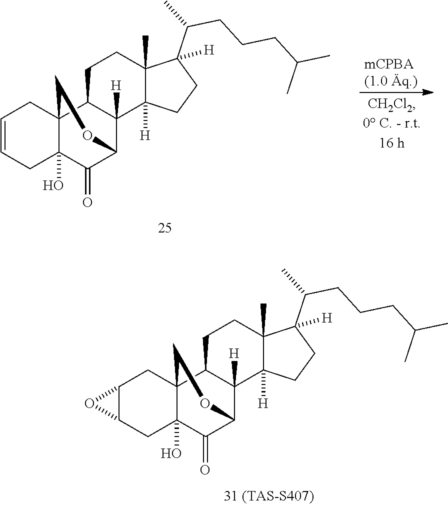

To a solution of 117 mg (0.28 mmol, 1.0 eq.) of alkene 25 in 5.0 mL of dichloromethane was added 58 mg (0.34 mmol, 1.2 eq.) of mCPBA at 0° C. The reaction mixture was stirred at room temperature for 16 h, and subsequently, the reaction was stopped by adding water. The aqueous phase was extracted with dichloromethane, and the combined organic phases were washed with saturated Na₂SO₃, NaHCO₃ and NaCl solutions. Subsequently, the combined organic phases were dried over MgSO₄, and the solvent was removed under reduced pressure. The residue was purified by column chromatography on silica (3:1, cHex/EtOAc), and the epoxide 31 could be obtained in the form of a colorless solid (95 mg, 0.22 mmol, 79%).

Analytics relating to 31:

Melting point: 189° C.-191° C.

Rf: 0.20 (2:1, cHex:EtOAc).

¹H NMR: (500 MHz, CDCl₃) δ [ppm]: 3.96 (d, J=10.1 Hz, 1H), 3.77 (d, J=1.6 Hz, 1H), 3.68 (dd, J=10.1, 1.6 Hz, 1H), 3.52 (s, 1H), 3.46 (dt, J=3.8, 1.9 Hz, 1H), 3.31 (dd, J=5.6, 3.8 Hz, 1H), 2.52-2.40 (m, 2H), 2.10-2.03 (m, 3H), 1.86 (dtd, J=13.2, 9.4, 6.2 Hz, 1H), 1.68-1.56 (m, 3H), 1.50 (dq, J=13.2, 6.6 Hz, 1H), 1.45-1.22 (m, 8H), 1.19-1.04 (m, 5H), 1.01-0.94 (m, 1H), 0.89 (d, J=6.5 Hz, 3H), 0.86 (dd, J=6.6, 2.5 Hz, 6H), 0.74 (s, 3H).

¹¹C NMR: (126 MHz, CDCl₃) δ [ppm]: 209.29, 77.72, 77.56, 65.18, 55.67, 54.11, 51.71, 51.25, 45.36, 44.75, 43.72, 40.05, 39.95, 39.56, 36.18, 35.79, 29.57, 28.62, 28.11, 23.92, 23.61, 22.93, 22.81, 22.67, 21.27, 18.72, 13.01.

FT-IR ν [cm⁻¹]=3460 (w), 2977 (m), 2943 (m), 2932 (m), 2922 (m), (ATR): 2864 (m), 1746 (s), 1466 (m), 1435 (w), 1382 (w), 1374 (m), 1326 (w), 1304 (w), 1264 (w), 1245 (w), 1234 (w), 1142 (m), 1130 (m), 1096 (m), 1051 (s), 1034 (m), 978 (w), 937 (m), 903 (s), 870 (m), 803 (s), 792 (m), 776 (m), 750 (m), 723 (m), 681 (w), 651 (w), 610 (m), 590 (m), 566 (m), 487 (w), 470 (m), 450 (s), 414 (w).

HR-MS: (ESI, 70 eV)=m/z calculated for: C₂₇H₄₃O₄[M+H]⁺ 431.315586 u, C₂₇H₄₂O₄Na [M+Na]⁺ 453.297531 u, found: 431.31568 u, 453.29764 u.

[α]_{λ}²⁰: c=0.602 g/100 mL, CHCl₃:
[α]₃₆₅²⁰=375.97° (±0.08°),
[α]₄₃₆²⁰=161.24° (±0.08°),
[α]₅₄₆=81.39° (±0.13°),
[α]₅₇₉²⁰=70.10° (±0.00°),
[α]₅₈₉²⁰=66.61° (±0.00°),

### Synthesis of 3β-acetoxy-19-hydroxy-stigmast-Δ⁵-ene (33) [WIL-DK04]

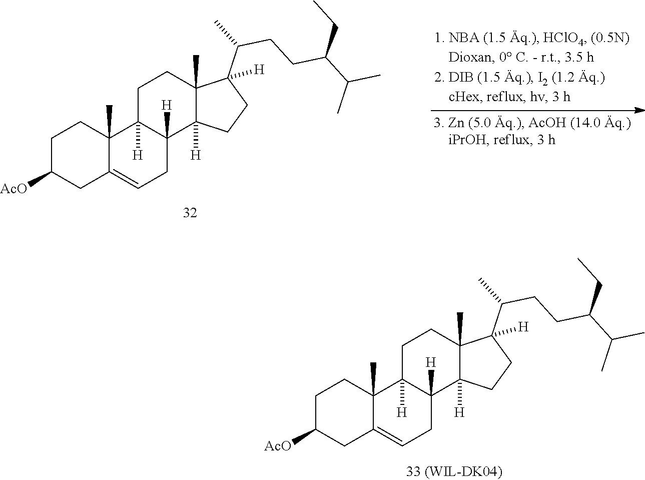

7.49 g (16.4 mmol, 1.0 eq.) of sitosteryl acetate (32) in 200 ml of dioxane was admixed with 3.39 g (23.6 mol, 1.5 eq.) of N-bromoacetamide (NBA) and 40 ml of HClO₄ (0.5 N). After 1 h at 0° C. in the dark and 2.5 h at room temperature, the reaction was stopped by adding a saturated Na₂SO₃ solution (until the reaction mixture changes its color) and water. After extraction with MTBE, the combined organic phases were washed with saturated NaCl solution and dried over MgSO₄. Subsequently, the solvent was removed under reduced pressure. The raw product (beige solid, 9.06 g), with considerable amounts (>40%) of regio-isomeric bromohydrin, was employed in the following step without further purification.

To a solution of the (about 9.06 g) bromohydrin (+regioisomer) from the previous raw product in 500 ml of cyclohexane were added 7.92 g (24.5 mmol, 1.5 eq.) of diacetoxyiodobenzene (DIB) and 4.99 g (19.6 mmol, 1.2 eq.) of iodine. The reaction mixture was heated under reflux for 3 h under irradiation by a 150 Watt mercury vapor lamp. The purple reaction mixture was cooled to room temperature, and quenched by adding a saturated Na₂SO₃ solution and water (until the color changes). After extraction with MTBE, the combined organic phases were washed with water and dried over MgSO₄. Subsequently, the solvent was removed under reduced pressure, and the raw product (brown viscous oil, 13.6 g) could be employed in the next step without further purification.

To a solution of the raw product from the previous reaction (13.6 g) in 400 ml of i-PrOH were added 5.35 g (8.19 mol, 5.0 eq.) of zinc powder and 13.8 mL (98 g, 22.9 mol, 14.0 eq.) of acetic acid. The reaction mixture was heated under reflux for 3 h, then cooled to room temperature, and filtered through Celite©. The clear yellow solution was concentrated to a volume of 100 mL, and extracted with MTBE after the addition of water. The combined organic phases were washed with water and a saturated NaCl solution, and subsequently dried over MgSO₄. The solvent was removed under reduced pressure. This procedure was performed for a total of four times. The product 33 was obtained after purification by column chromatography on silica gel (c-hex/EtOAc, 4:1) in the form of a beige solid (2.67 g, 5.82 mol, 35% over three stages).

Analytics relating to 33:

Melting point: 115° C.-118° C.

Rf: 0.33 (4:1, cHex:EtOAc).

¹H NMR: (500 MHz, CDCl₃) δ [ppm]: 5.78 (dd, J=4.8, 2.4 Hz, 1H), 4.64 (tt, J=11.4, 4.8 Hz, 1H), 3.83 (d, J=11.4 Hz, 1H), 3.62 (d, J=11.4 Hz, 1H), 2.42 (ddd, J=13.0, 4.9, 2.3 Hz, 1H), 2.31-2.23 (m, 1H), 2.07-1.99 (m, 5H), 1.96 (dt, J=13.9, 3.7 Hz, 1H), 1.90-1.77 (m, 3H), 1.70-1.46 (m, 7H), 1.40-0.98 (m, 11H), 0.95-0.89 (m, 6H), 0.86-0.80 (m, 9H), 0.80-0.76 (m, 1H), 0.73 (s, 3H).

¹³C NMR: (126 MHz, CDCl₃) δ [ppm]: 170.65, 134.66, 128.47, 73.55, 62.87, 57.71, 56.14, 50.45, 45.97, 42.67, 41.75, 40.13, 38.36, 36.28, 34.07, 33.52, 33.24, 31.39, 29.28, 28.39, 28.25, 26.21, 24.23, 23.20, 21.91, 21.54, 19.96, 19.17, 18.91, 12.36, 12.12.

FT-IR ν [cm⁻¹]=3497 (bs), 2956 (s), 2932 (s), 2866 (s), 1728 (m), (ATR): 1464 (s), 1441 (S), 1377 (m), 1255 (m), 1133 (s), 1088 (s), 1033 (m), 960 (s), 916 (s), 883 (s), 841 (s), 810 (s), 798 (s), 623 (s), 585 (s).

HR-MS: (ESI, 70 eV)=m/z calculated for: C₃₁H₅₂O₃Na [M+Na]⁺481.365217 u, found: 481.36525 u.

[α]_{λ}²⁰: c=0.95 g/100 mL, CHCl₃:
[α]₃₆₅²⁰=−87.44° (±0.44°),
[α]₄₃₆²⁰=−53.19° (±0.13°),
[α]₅₄₆²⁰=−30.46° (±0.05°),
[α]₅₇₉²⁰=−27.02° (±0.05°),
[α]₅₈₉²⁰=−26.95° (±0.00°),

### Synthesis of 3μ-acetoxy-19-(methoxymethyloxy)stigmastan-Δ⁵-ene (34) [WIL-DK05]

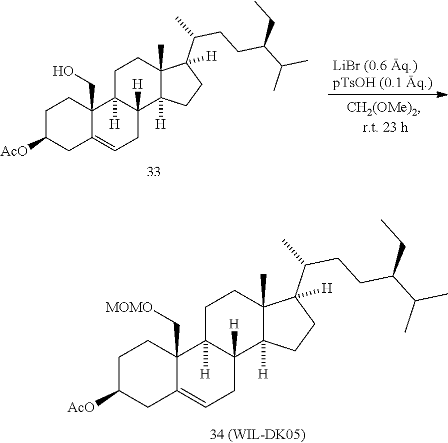

2.67 g (5.82 mmol, 1.0 eq.) of 19-hydroxystigmastyl acetate (33) in 34 ml of CH₂(OMe)₂ was admixed with 300 g (2.31 mmol, 0.6 eq.) of lithium bromide and 0.11 g (0.57 mmol, 0.1 eq.) of p-toluenesulfonic acid monohydrate, and the reaction mixture was stirred at room temperature for 23 h. After complete conversion was observed (TLC control), the reaction was stopped by adding water, and the aqueous phase was extracted with MTBE. The combined organic phases were washed with water and a saturated NaCl solution, and dried over MgSO₄. The product 34 could be obtained after purification by column chromatography on silica gel (c-hex/EtOAc, 10:1) in the form of a brown solid (2.37 g, 4.59 mol, 79%).

Analytics relating to 34:

Melting point: 83° C.-84° C.

Rf: 0.39 (10:1, cHex:EtOAc).

¹H NMR: (500 MHz, CDCl₃) δ [ppm]: 5.60 (dt, J=4.4, 1.9 Hz, 1H), 4.69-4.57 (m, 3H), 3.73 (d, J=10.3 Hz, 1H), 3.48 (d, J=10.3 Hz, 1H), 3.37 (s, 3H), 2.40 (ddd, J=13.1, 5.2, 2.2 Hz, 1H), 2.36-2.27 (m, 1H), 2.19-2.15 (m, 1H), 2.11 (dt, J=13.5, 3.5 Hz, 1H), 2.07-1.96 (m, 5H), 1.84 (dtd, J=15.5, 9.2, 6.0 Hz, 2H), 1.79-1.61 (m, 2H), 1.64-1.45 (m, 6H), 1.39-0.98 (m, 15H), 0.97-0.86 (m, 6H), 0.88-0.79 (m, 9H), 0.81-0.75 (m, 1H), 0.70 (s, 3H).

¹³C NMR: (126 MHz, CDCl₃) δ [ppm]: 170.67, 135.81, 126.26, 97.05, 73.83, 69.07, 57.42, 56.14, 55.60, 50.47, 45.99, 42.59, 40.52, 40.18, 38.50, 36.31, 34.09, 33.45, 32.91, 31.73, 29.29, 28.40, 28.19, 26.21, 24.35, 23.21, 21.99, 21.57, 19.96, 19.18, 18.91, 12.19, 12.13.

FT-IR ν [cm⁻¹]=2952 (bs), 2933 (s), 2869 (s), 1728 (m), 1463 (s), (ATR): 1443 (s), 1379 (m), 1369 (s), 1243 (m), 1140 (s), 1112 (s), 1045 (m), 1027 (m), 917 (s), 916 (s), 881 (s), 839 (s), 802 (s), 736 (s), 611 (s), 578 (s).

HR-MS: (ESI, 70 eV)=m/z calculated for: C₃₃H₅₆O₄Na [M+Na]⁺539.407081 u, found: 539.40665 u.

[α]_{λ}²⁰: c=0.85 g/100 mL, CHCl₃:
[α]₃₆₅²⁰=−87.44° (±0.44°),
[α]₄₃₆²⁰=−53.19° (±0.13°),
[α]₅₄₆²⁰=−30.46° (±0.05°),
[α]₅₇₉²⁰=−27.02° (±0.05°),
[α]₅₈₉²⁰=−26.95° (±0.00°),

### Synthesis of 3β-acetoxy-5α-hydroxy-19-(methoxymethyloxy)-sitgmastan-6-one (35) [WIL-DK06]

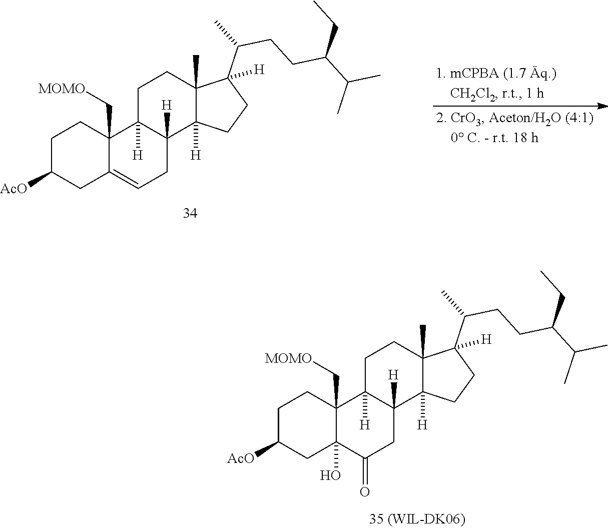

2.27 g (4.39 mmol, 1.0 eq.) of compound 34 was dissolved in 33 ml of CH₂Cl₂, and admixed with 1.28 g (7.47 mmol, 1.7 eq.) of mCPBA (70%). The reaction mixture was stirred at room temperature for 1 h and then cooled down to 0° C. The white precipitate formed was dissolved by adding 81 mL of acetone, and the clear solution was admixed with a solution of 2.37 g (23.4 mmol, 5.4 eq.) of CrO₃ in 8.0 mL of water. After 10 minutes at 0° C., the reaction mixture was taken to room temperature, and stirred for 18 h. Subsequently, the reaction mixture was stopped by adding a saturated NaHCO₃ solution, and the aqueous phase was extracted with EtOAc. The combined organic phases were washed with water and a saturated NaCl solution. The clear yellow solution was dried over MgSO₄, and the solvent was removed under reduced pressure. Subsequently, the desired product 35 was obtained after purification by column chromatography on silica gel (c-hex/EtOAc, 3:1) in the form of a colorless gel (1.51 g, 2.76 mol, 61%).

Analytics relating to 35:

Rf: 0.19 (3:1, cHex:EtOAc).

¹H NMR: (500 MHz, CDCl₃) δ [ppm]: 5.11 (tt, J=11.2, 5.3 Hz, 1H), 4.50 (s, 2H), 3.64 (d, J=10.7 Hz, 1H), 3.57 (d, J=10.6 Hz, 1H), 3.34 (s, 3H), 2.83 (s, 1H), 2.57 (dd, J=14.6, 11.5 Hz, 1H), 2.21-2.12 (m, 2H), 2.08-1.99 (m, 5H), 1.96-1.82 (m, 3H), 1.77-1.63 (m, 3H), 1.59-1.43 (m, 4H), 1.39-0.98 (m, 12H), 0.92 (d, J=6.4 Hz, 4H), 0.88-0.76 (m, 10H), 0.68 (s, 3H).

¹³C NMR: (126 MHz, CDCl₃) δ [ppm]: 209.63, 171.09, 97.12, 77.83, 70.46, 67.13, 57.05, 56.16, 56.05, 45.92, 45.70, 44.09, 43.22, 41.54, 39.99, 37.20, 36.26, 34.01, 32.48, 29.26, 28.31, 26.56, 26.28, 26.09, 24.05, 23.19, 21.93, 21.50, 19.95, 19.16, 18.84, 12.25, 12.11.

FT-IR ν [cm⁻¹]=3417 (bs), 2936 (m), 2870 (s), 1713 (m), 1463 (s), (ATR): 1443 (s), 1379 (m), 1365 (s), 1236 (m), 1150 (s), 1106 (s), 1034 (m), 1012 (s), 967 (s), 941 (s), 919 (s), 835 (s), 666 (s), 608 (s), 581 (s), 552 (s).

HR-MS: (ESI, 70 eV)=m/z calculated for: C₃₃H₅₆O₆Na [M+Na]⁺571.396911 u, found: 571.39677 u.

[α]_{λ}²⁰: c=0.90 g/100 mL, CHCl₃:
[α]₃₆₅²⁰=−243.15° (±0.14°),
[α]₄₃₆²⁰=−116.29° (±0.14°),
[α]₅₄₆²⁰=−60.18° (±0.05°),
[α]₅₇₉²⁰=−51.96° (±0.05°),
[α]₅₈₉²⁰=−49.93° (±0.10°),

### Synthesis of 3β-acetoxy-7α-bromo-5α,6α-dihydroxy-6β,19-epoxy-stigmastan (36) [WIL-DK07]

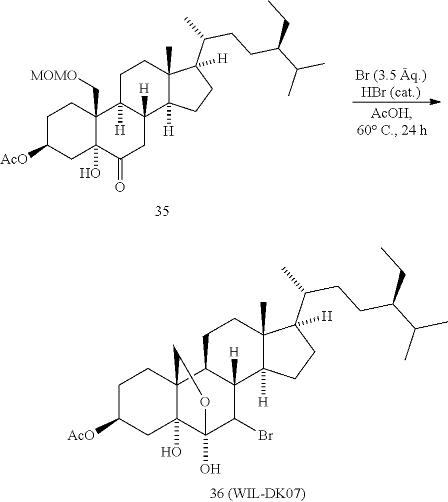

To a solution of 1.34 g (2.45 mmol, 1.0 eq.) of compound 35 in 25 mL of acetic acid were added 1.37 mL (8.57 mmol, 3.5 eq.) of bromine and 8 drops of HBr (48% aqu.). The reaction mixture was heated at 60° C. for 24 h. Subsequently, the reaction mixture was cooled to room temperature, and quenched with a saturated Na₂SO₃ solution. The aqueous phase was extracted with EtOAc, the combined organic phases were washed with water and saturated NaCl solution, and dried over MgSO₄. Then, the solvent was removed under reduced pressure, and the product 36 was obtained after purification by column chromatography in the form of a white solid (657 mg, 1.13 mmol, 61%).

Analytics relating to 36:

Melting point: 93° C.-94° C.

Rf: 0.21 (5:1, cHex:EtOAc).

¹H NMR: (500 MHz, CDCl₃) δ [ppm]: 5.03 (tt, J=11.0, 4.6 Hz, 1H), 4.13 (d, J=4.7 Hz, 1H), 4.06-4.01 (m, 1H), 3.73 (d, J=8.9 Hz, 1H), 2.20 (ddd, J=12.9, 4.4, 2.2 Hz, 1H), 2.10 (s, 1H), 2.07-2.00 (m, 4H), 1.97-1.83 (m, 5H), 1.75 (dd, J=12.9, 11.9 Hz, 1H), 1.70-1.64 (m, 1H), 1.60-1.00 (m, 17H), 0.96-0.89 (m, 4H), 0.88-0.76 (m, 10H), 0.75 (s, 3H).

¹³C NMR: (151 MHz, CDCl₃) δ [ppm]: 170.81, 101.79, 79.34, 69.92, 66.86, 59.35, 55.56, 52.87, 45.94, 45.37, 43.44, 38.98, 38.64, 38.53, 36.21, 35.01, 34.01, 29.29, 28.11, 27.18, 26.22, 24.01, 23.28, 23.21, 21.57, 21.54, 19.99, 19.15, 18.85, 13.12, 12.12.

FT-IR ν [cm¹]=3413 (bs), 2954 (m), 2936 (m), 2869 (s), 2050 (s), (ATR): 1713 (m), 1497 (s), 1457 (s), 1377 (m), 1365 (s), 1243 (m), 1153 (s), 1129 (s), 1036 (m), 1095 (s), 985 (s), 945 (s), 906 (s), 844 (s), 702 (s), 677 (s), 628 (s), 608 (s), 528 (s).

HR-MS: (GC-EI/MS, 70 eV, 50-250° C.) m/z (%)=81.06980 (88), 109.06468 (100), 255.17394 (55), 396.33803 (30), 414.34872 (45), 474.36932 (10), 502.36426 (<5).

[α]_{λ}²⁰: c=1.00 g/100 mL, CHCl₃:
[α]₃₆₅²⁰=−137.92° (±3.68°),
[α]₄₃₆²⁰=−84.96° (±0.12°),
[α]₅₄₆²⁰=−50.07° (±0.21°),
[α]₅₇₉²⁰=−43.78° (±0.12°),
[α]₅₈₉²⁰=−42.25° (±0.28°).

### Synthesis of 3β-acetoxy-5α-hydroxy-7β,19-epoxy-stigmastan-6-one (37) [WIL-DK08]

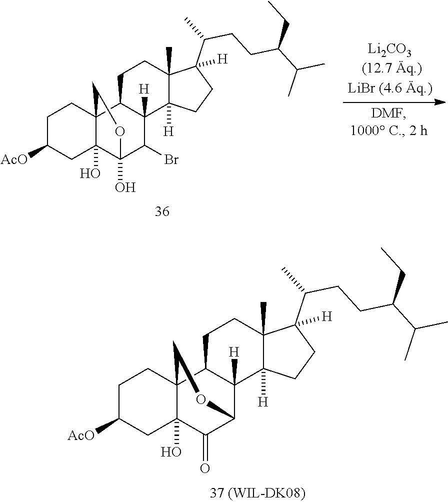

533 mg (0.913 mmol, 1.0 eq.) of compound 36 was dissolved in 46 ml of dimethylformamide and admixed with 858 mg (11.6 mmol, 12.7 eq.) of Li₂CO₃ and 365 mg (4.20 mmol, 4.6 eq.) of LiBr. The reaction mixture was stirred at 100° C. for 2 h. Subsequently, the reaction mixture was cooled to room temperature, and diluted with water. The aqueous phase was extracted with EtOAc, the combined organic phases were washed with water and saturated NaCl solution, and dried over MgSO₄. Subsequently, the solvent was removed under reduced pressure, and the product 37 was obtained after purification by column chromatography (c-Hex/EtOAc, 2.5:1) in the form of a white solid (321 mg, 0.639 mmol, 54%).

Analytics relating to 37:

Melting point: 142° C.-143° C.

Rf: 0.33 (2.5:1, cHex:EtOAc).

¹H NMR: (500 MHz, CDCl₃) δ [ppm]: 5.22 (tt, J=11.2, 5.5 Hz, 1H), 4.14 (dd, J=10.3, 1.7 Hz, 1H), 3.91 (d, J=10.2 Hz, 1H), 3.82 (d, J=1.7 Hz, 1H), 2.63 (s, 1H), 2.13-2.01 (m, 6H), 2.00-1.93 (m, 2H), 1.88 (dtd, J=13.0, 9.4, 5.9 Hz, 1H), 1.77-1.49 (m, 5H), 1.42-1.11 (m, 14H), 1.05-0.97 (m, 1H), 0.95-0.87 (m, 4H), 0.87-0.75 (m, 9H), 0.74 (s, 3H).

¹³C NMR: (126 MHz, CDCl₃) δ [ppm]: 212.88, 170.55, 78.65, 76.17, 69.18, 63.55, 55.60, 52.02, 45.94, 45.52, 45.09, 43.21, 40.53, 40.23, 36.13, 35.23, 33.98, 29.24, 28.66, 26.22, 26.11, 23.18, 22.90, 21.70, 21.46, 21.25, 19.93, 19.14, 18.80, 13.07, 12.11.

FT-IR ν [cm⁻¹]=3533 (bs), 2953 (m), 2863 (s), 2112 (s), 1733 (m), (ATR): 1499 (s), 1463 (s), 1375 (m), 1332 (s), 1253 (m), 1151 (s), 1097 (s), 1042 (m), 1024 (s), 963 (s), 945 (s), 910 (s), 892 (s), 870 (s), 753 (s), 710 (s), 655 (s), 635 (s), 606 (s), 574 (s).

HR-MS: (GC-EI/MS, 70 eV, 50-250° C.) m/z (%)=81.06982 (100), 109.06469 (100), 255.17406 (65), 396.33804 (35), 414.34868 (50), 474.36956 (10), 502.36451 (<1).

[α]_{λ}²⁰: c=0.91 g/100 mL, CHCl₃:
[α]₃₆₅²⁰=111.64° (±0.27°),
[α]₄₃₆²⁰=66.74° (±0.16°),
[α]₅₄₆²⁰=39.38° (±0.14°),
[α]₅₇₉²⁰=34.36° (±0.16°),
[α]₅₈₉²⁰=33.11° (±0.14°).

### Synthesis of 3β,5α-dihydroxy-7β,19-epoxy-stigmastan-6-one (38) [WIL-DK09]

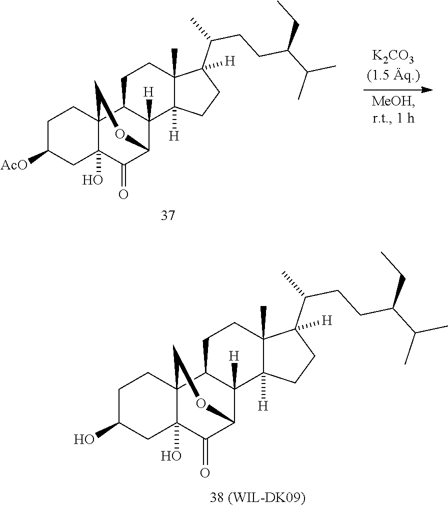

To a solution of 222 mg (0.442 mmol, 1.0 eq.) of 37 in 30 mL of methanol was added 91 mg (0.66 mmol, 1.5 eq.) of K₂CO₃, and the reaction mixture was stirred at room temperature for 1 h. After the reaction was complete (TLC), the reaction mixture was diluted with water. The aqueous phase was extracted with EtOAc, the combined organic phases were washed with NaCl solution, and dried over MgSO₄. Subsequently, the solvent was removed under reduced pressure, the residue was purified by column chromatography (c-Hex/EtOAc, 1:1) any by recrystallization from EtOH/H₂O. The product 38 was obtained in the form of a white solid (110 mg, 0.685 mmol, quant.).

Analytics relating to 38:

Melting point: 175° C.-176° C.

Rf: 0.17 (1:1, cHex:EtOAc).

¹H NMR: (500 MHz, CDCl₃) δ [ppm]: 4.22-4.12 (m, 2H), 3.89 (d, J=10.2 Hz, 1H), 3.82 (d, J=1.7 Hz, 1H), 2.14-2.01 (m, 3H), 1.97-1.84 (m, 3H), 1.71-1.54 (m, 4H), 1.48 (tdd, J=13.4, 11.2, 4.9 Hz, 1H), 1.42-1.11 (m, 14H), 1.05-0.98 (m, 1H), 0.94-0.88 (m, 4H), 0.86-0.75 (m, 10H), 0.74 (s, 3H).

¹³C NMR: (126 MHz, CDCl₃) δ [ppm]: 213.65, 79.18, 76.18, 66.23, 63.66, 55.62, 52.06, 45.93, 45.53, 45.19, 43.31, 40.59, 40.28, 38.87, 36.12, 33.99, 30.19, 29.24, 28.67, 26.10, 23.18, 22.91, 21.76, 21.52, 19.93, 19.14, 18.80, 13.07, 12.11.

FT-IR ν [cm⁻¹]=3353 (bs), 2955 (m), 2861 (s), 2050 (s), 1732 (m), (ATR): 1495 (s), 1463 (s), 1376 (m), 1365 (s), 1250 (m), 1225 (s), 1154 (s), 1070 (s), 1049 (m), 969 (s), 914 (s), 88 (s), 872 (s), 853 (s), 818 (s), 754 (s), 710 (s), 6635 (s), 637 (s), 583 (s), 559 (s).

HR-MS: (GC-EI/MS, 70 eV, 50-300° C.) m/z (%)=81.06982 (100), 109.06472 (60), 133.10107 (55), 273.18460 (35), 414.34881 (25), 432.35897 (15), 460.35410 (<1).

[α]_{λ}²⁰: c=0.86 g/100 mL, CHCl₃:
[α]₃₆₅²⁰=248.03° (±0.20°),
[α]₄₃₆²⁰=120.94° (±0.34°),
[α]₅₄₆²⁰=63.82° (±0.58°),
[α]₅₇₉²⁰=56.53° (±0.54°),
[α]₅₈₉²⁰=53.76° (±0.71°),

### Example 2: Inhibition of Proliferation in Leukemia Cells (Nalm-6 Cells)

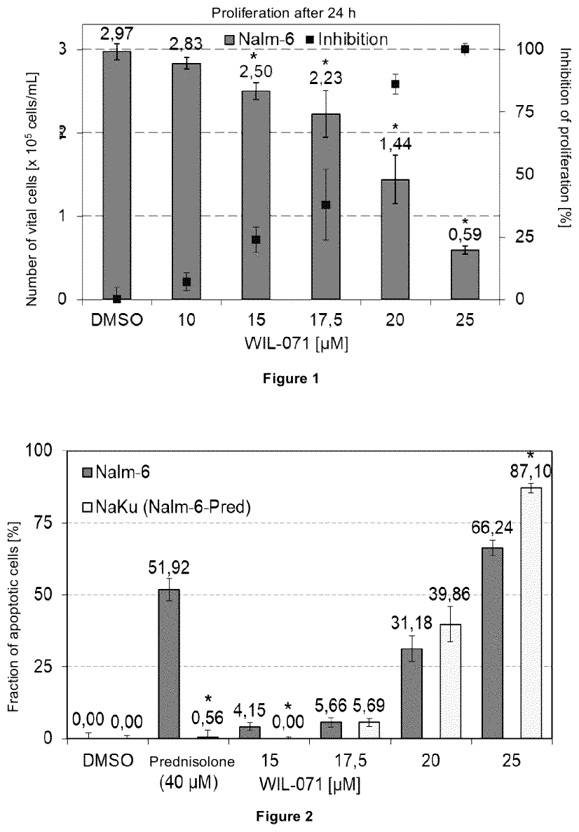

In leukemia cells (Nalm-6), cell proliferation is inhibited. After 24 h, 1×10⁵ cells will have proliferated to almost threefold (2.97×10⁵ cells), while cell proliferation is inhibited up to 100% in a concentration-dependent way by treatment with the steroid WIL 071, see FIG. 1. The experiments were performed in triplicate. The error bars show the standard deviations of the measured values of three independent experiments.

### Example 3: Overcoming Steroid and Polycytostatics Resistances in NaKu Cells

In steroid- and polycytostatics-resistent leukemia cells (NaKu (ALL)), treatment with the steroid WIL 071 induces as much apoptosis as in the non-resistent leukemia cells (Nalm-6) in a non-concentration-dependent way, see FIG. 2. The experiments were performed in triplicate. The error bars show the standard deviations of the measured values of three independent experiments.

The steroid WIL 071 overcomes steroid resistence in leukemia cells (NaKu cells). Further, the steroid-resistent leukemia cells (NaKu cells) have co-resistencies for the following cytostatics:
prednisolone, methylprednisolone, dexamethasone,
doxorubicin, daunorubicin, idarubicin, epirubicin
cytarabine, cladribine
etoposide
cyclophosphamide, ifosfamide
vindesine
cisplatin, carboplatin, oxaliplatin

### Example 4: Overcoming of Polycytostatics Resistance in BiBo Cells

BiBo cells are lymphoma cells (BJAB cells) that have been made resistent against vincristine, and have a Bcl-2 overexpression as a resistence mechanism. They have co-resistences for the following cytostatics:
vincristine, vinblastine, vindesine, vinorelbin, taxol
cytarabin
daunorubicin.

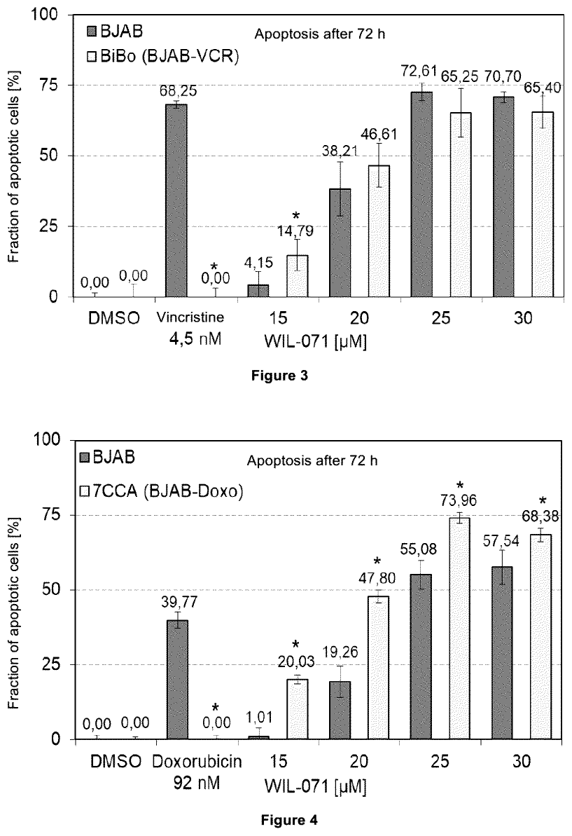

In polycytostatics-resistent lymphoma cells (BiBo cells), treatment with the steroid WIL 071 induces as much apoptosis as in the non-resistent lymphoma cells (BJAB (Burkitt like lymphoma cells)) in a non-concentration-dependent way, see FIG. 3. The experiments were performed in triplicate. The error bars show the standard deviations of the measured values of three independent experiments. The steroid WIL 071 overcomes steroid resistence in lymphoma cells (BiBo cells).

### Example 5: Overcoming of Polycytostatics Resistance in 7-CCA Cells

7-CCA cells are lymphoma cells (BJAB cells) that have been made resistent against doxorubin, and have a caspase-3 underexpression as a resistence mechanism. They have co-resistences for the following cytostatics:
vincristine, vinblastine, vindesine, vinorelbin, taxol
cytarabin, fudarabin, cladribin, clofarabin,
daunorubicin, doxorubicin, idarubicin, epirubicin
cisplatin, oxaliplatin

In polycytostatics-resistent lymphoma cells (7-CCA cells), treatment with the steroid WIL 071 induces as much apoptosis as in the non-resistent lymphoma cells (BJAB (Burkitt like lymphoma cells)) in a non-concentration-dependent way, see FIG. 4. The experiments were performed in triplicate. The error bars show the standard deviations of the measured values of three independent experiments. The steroid WIL 071 overcomes steroid resistence in lymphoma cells (7-CCA cells).

### Example 6: Synergistic Effects with Cytarabin and Vincristine

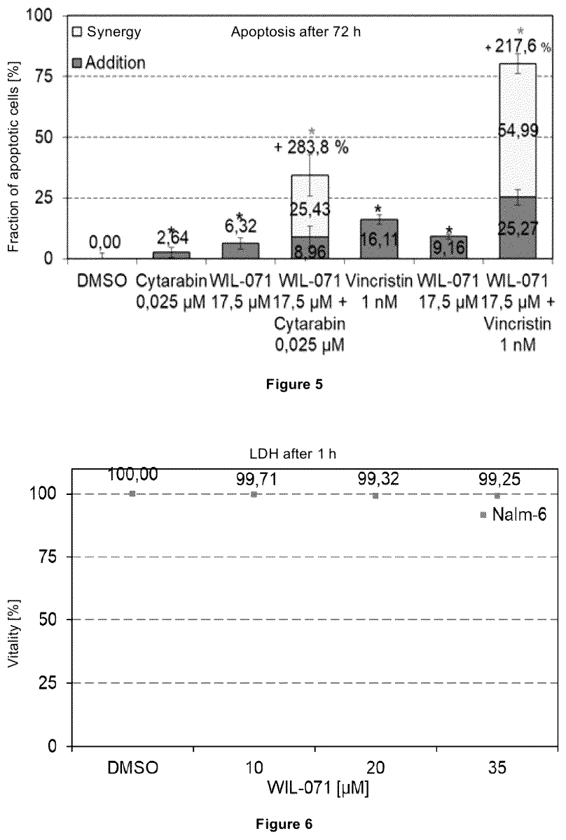

In leukemia cells (Nalm-6 cells), parallel treatment with the steroid WIL 071 and cytarabin or vincristine induces significantly more apoptosis than 1.1 times the sum of the apoptotic rates in treatment by a monotherapy with the corresponding drugs, see FIG. 5. The experiments were performed in triplicate. The error bars show the standard deviations of the measured values of three independent experiments. The steroid WIL 071 has synergistic effects with cytarabin and vincristine.

### Example 7: Exclusion of Non-Specific Necrosis

What is measured is the release of lactate dehydrogenase (LDH) in leukemia cells (Nalm-6 cells) in the cell supernatant, which is an indication of non-specific cell death (necrosis), because this large protein LDH can cross cell membranes towards the extracellular space only if they are defect, see FIG. 6. Non-specific necrosis can be excluded.

### Example 8: Selectivity

In leukemia cells (Nalm-6 cells) and in lymphoma cells (BJAB cells), apoptosis is induced in a concentration-dependent way by treatment with the steroid WIL 071. WIL 071 acts selectively in malignant cells that are proliferating. In contrast, apoptosis is not induced in human primary leukocytes.

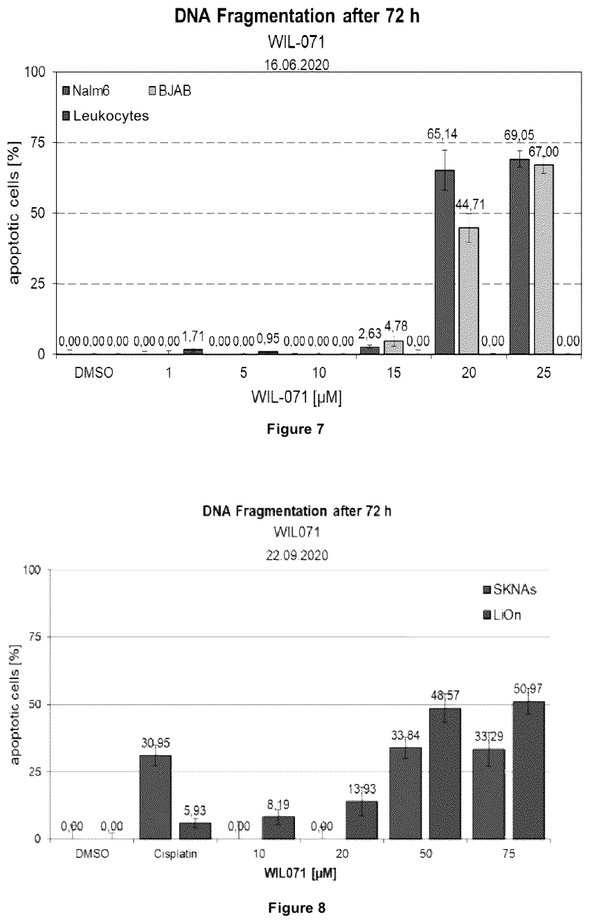

The DNA fragmentation was measured by flow cytometry, see FIG. 7. The experiments were performed in triplicate. The error bars show the standard deviations of the measured values of three independent experiments.

### Example 9: Effect in Cytostatics-Resistant Solid Tumors

In contrast to conventional glucocorticoids, the steroid WIL 071 can induce apoptosis also in solid tumor cells. Resistances towards cytostatics are overcome also in neuroblastoma cells (SKNAS cells) (LiOn cells—cisplatin-resistant SKNAS cells with an underexpression of caspase-8.

The DNA fragmentation was measured by flow cytometry), see FIG. 8. The experiments were performed in triplicate. The error bars show the standard deviations of the measured values of three independent experiments.

### Example 10: Effect in Brain Tumor Cells

In contrast to conventional glucocorticoids, the steroid WIL 071 can induce apoptosis also in brain tumor cells. The steroid WIL 071 significantly induces apoptosis also in glioblastoma cells (DBTRG05MG cells).

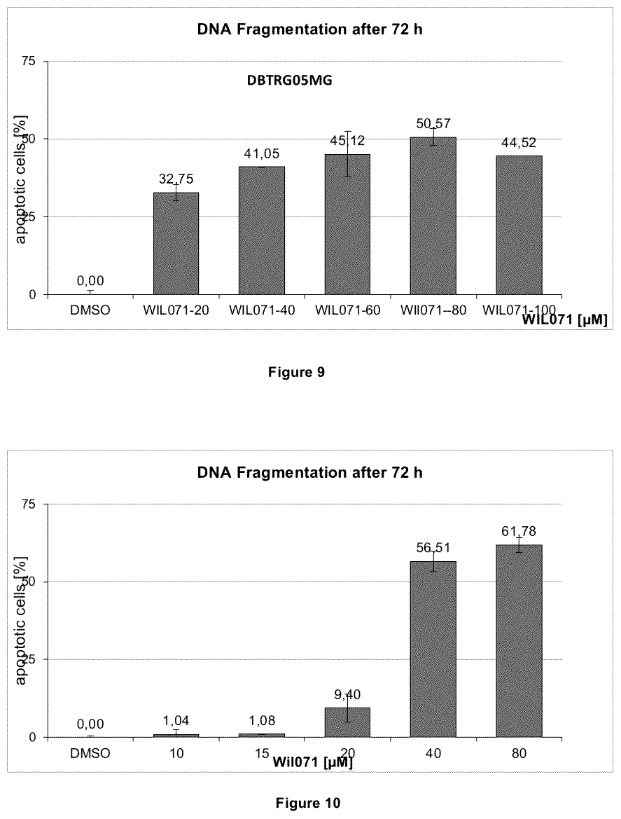

The DNA fragmentation was measured by flow cytometry, see FIG. 9. The experiments were performed in triplicate. The error bars show the standard deviations of the measured values of three independent experiments.

### Example 11: Effect in Primary Human Glioblastoma Cells

In contrast to conventional glucocorticoids, the steroid WIL 071 can induce apoptosis also in primary human brain tumor cells. The steroid WIL 071 significantly induces apoptosis also in primary glioblastoma cells of a patient having a glioblastoma (grade 4).

The DNA fragmentation was measured by flow cytometry, see FIG. 10. The experiments were performed in triplicate. The error bars show the standard deviations of the measured values of three independent experiments.

### Example 12: Effect on Pancreatic Carcinoma Cells

In contrast to conventional glucocorticoids, the steroid WIL 071 can induce apoptosis also in pancreatic carcinoma cells. In a corresponding cell line (DAN-G), the steroid WIL 071 significantly induces apoptosis.

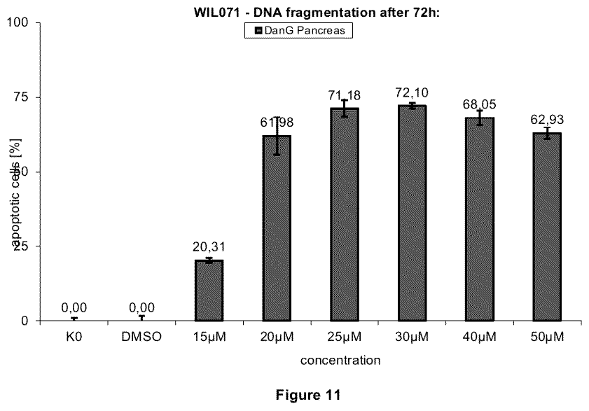

The DNA fragmentation was measured by flow cytometry, see FIG. 11. The experiments were performed in triplicate. The error bars show the standard deviations of the measured values of three independent experiments.

The standard treatment for pancreatic carcinoma includes 5-fluorouracil. It shows clearly worse apoptosic rates in comparable experiments (Oxaliplatin: 14% apoptosis at 50 μM, 5-fluorouracil 24.9% at 30 μM, 21.2% at 50 μM).

### Example 13: Comparison of the Biological Effects of Different Steroid Derivatives in Leukemia Cells (Nalm-6)

| | Biological effect: half- | Overcoming of |
|---|---|---|
| Steroid | maximal apoptosis induction | prednisolone |
| derivative | (AC₅₀) [μM] | resistance [μM] |
| WIL-071 | 17.2 | 16 |
| WIL-232 | 25 | 17.5 |
| WIL-241 | 41 | |
| WIL-242b | 37 | 40 |
| WIL-270 | 20 | 18 |
| WIL357 | >50 | >50 |
| WIL360 | >20 | 20 |
| WIL364 | ~18 | ~17.5 |
| WIL367 | ~8 | ~12.5 |
| WIL369 | ~3 | 5 |
| WIL-DK-08 | 50 | ~40 |
| WIL-DK-09 | ~15 | ~38 |
| TAS-S389 | ~20 | ~30 |
| TAS-S392 | ~46 | ~39 |
| TAS-S398 | ~80 | ~67 |
| TAS-S399 | 50 | ~75 |
| TAS-S403 | 100 | 20 |
| TAS-S405 | 90 | ~20 |
| TAS-S406 | 13 | ~17 |

Apoptosis Induction in Nalm-6 Cells after Treatment with WIL-071 or Different Steroid Derivatives.

The cells were incubated in 6-well plates with 1.0·10⁵ cells/mL in two milliliters of medium with the respective steroid derivative in different concentrations. The included solvent control (DMSO) served as a control, which was subtracted from all the other measured values, in order not to take into account the spontaneous apoptosis of the cells that takes place independently of the concentration of active ingredient. After 72 h, a modified cell cycle analysis was performed, in order to quantify the proportion of apoptotic cells by flow cytometry. For each concentration of the steroid, the respective mean values f standard deviations, n=3 (*: p<0.05 vs. DMSO, t test) were determined. Using the curve of the dose-effect relationship (steroid concentration−apoptosis induction) formed from these measured values, the steroid concentration at which apoptosis was triggered in 50% of the tested leukemia cells was read.

Prednisolone resistance is said to be overcome if the induction of apoptosis in prednisolone-resistant leukemia cells is as high as it is in normal leukemia cells.

### Example 14: Examination of Tolerability in an Animal Model

An incremental increase of dose of the three substances Wil-071, Wil-232 and Wil-369 was performed in 8-week-old female NOG-F (Taconic) mice. Different mice were administered one of up to seven different doses of the test substances. The start was on day 1 with the lowest dose of 10 mg/kg. If no behavioural abnormalities or observable health symptoms showed in the course of the next 24 hours after such injection into the caudal vene of the animal, then the treatment was continued on the next day with the next higher dose. All in all, if possible, the doses 10, 20, 40, 80, 100, 150 and 200 mg/kg are tested. Subsequently, the maximum tolerable dose (MTD) was administered to three more animals, and these were subsequently observed for one week, in order to be able to observe any developing symptoms. For this purpose, the animals were observed and weighed on a daily basis. The body weight is considered an indicator of the general condition of the animals.

Following the determination of the MTD, eight animals were again injected the MTD of the substances, and after 1, 2, 8 and 24 hours, 2 animals each were killed. For pharmacokinetics, the liver, brain and serum of the animals were shock-frosted in liquid nitrogen, and analyzed (see Example 15).

### Results

All three substances are sparingly soluble in tested injection solutions, so that the maximal dose could not exceed 200 mg/kg.

For the MTD experiment, Wil-071 was dissolved in 100% DMSO, and then diluted 1:50 with a 20% (w/v) 2-hydroxypropyl-beta-cyclodextrin (HPβCD) solution. Thus, 200 μL of injection solution (per mouse) of a 40 mg/kg dose could be achieved. For 80, 100 and 150 mg/kg, the DMSO proportion was increased to up to 4% (v/v) in the final injection solution. In addition, ultrasound had to be employed, and the sample had to be heated at 60° C. for up to 45 min. 200 mg/kg was reached only as a milky suspension. The animals did not show any abnormalities after the injection of the individual doses, and the final section did not show any toxicity macroscopically. However, the injection of the 200 mg/kg suspension resulted in the immediate death of the animal. A section showed signs of pulmonary embolism. Therefore, Wil-071 can be considered non-toxic up to 150 mg/kg. The final MTD could not be determined because of the insufficient solubility.

For 100 mg/kg of Wil-232, the end concentration of DMSO in the injection solution was 2%. In order to dissolve Wil-232, the sample also had to be treated with ultrasound, and heated at 60° C. The dose of 150 mg/kg was achieved only with 4% DMSO, and 200 mg/kg was achieved only as a suspension. The injection of the 200 mg/kg dose resulted in the immediate death of the animal (signs of pulmonary embolism in section). Otherwise, there were no signs of toxicity, so that the substance can be considered tolerable up to 150 mg/kg. In this case too, a final MTD cannot be determined because of insufficient solubility.

Wil-369 showed the lowest solubility. Using a DMSO stock and 20% HPβCD solution, a maximum of only 5 mg/ml was dissolved. A further increase could not be achieved even with more DMSO or HPβCD. Thus, above the dose of 40 mg/kg, a higher injection volume was injected in order to reach the corresponding dose. With 100 mg/kg in twice as large injection volumes, the animals lay down on their side for some moments, but recovered quickly. In the further course and in the section, all animals were normal, except for one animal from the 100 mg/kg dose group, which was found dead on day 3 after the injection. For the tolerability under the current solubility conditions, a value of 50 mg/mg/kg is to be applied. This dose was injected into the 8 animals for pharmacokinetics. In this case too, a final MTD cannot be determined because of insufficient solubility.

### Recognition

In the concentration range that allows the substances to be dissolved completely, all substances are well tolerable.

### Example 15: Distribution

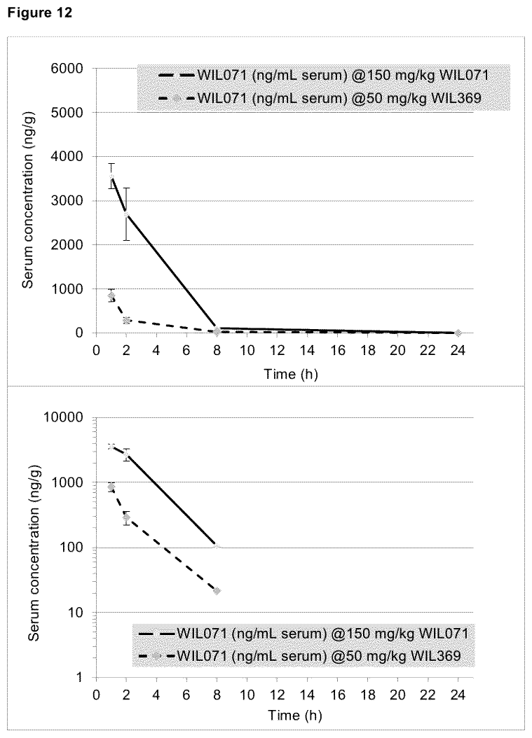

The samples obtained in Example 4 were examined for the contents of the substances. Since WIL-369 can be hydrolyzed to WIL-071, both WIL-369 and WIL-071 were determined in the samples. As far as these substances were detectable in the samples, this is represented in the Figures. The distribution is shown in FIGS. 12 to 14.

FIG. 12 shows that WIL-071 is formed when WIL-369 is administered, but the serum levels are cleared quickly; thus, WIL-369 is a prodrug.

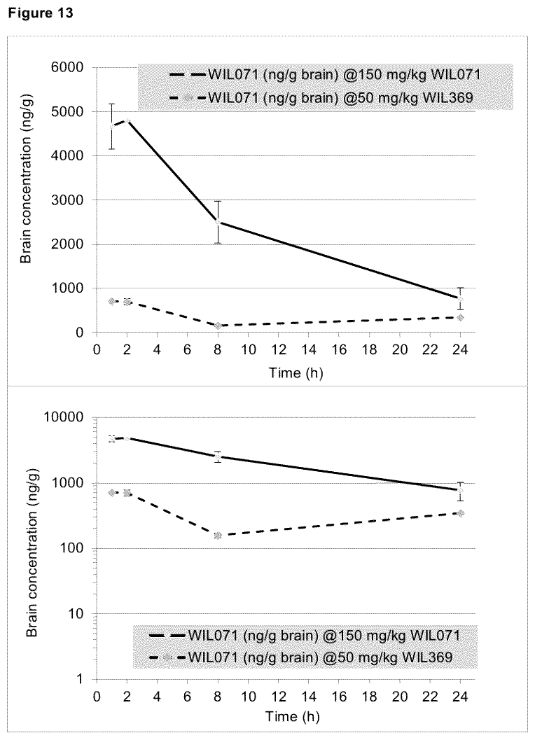

FIG. 13 shows relevant levels of WIL-071 after administration of WIL-369 or WIL-071 in the brain, which could be effective for more than 8 hours.

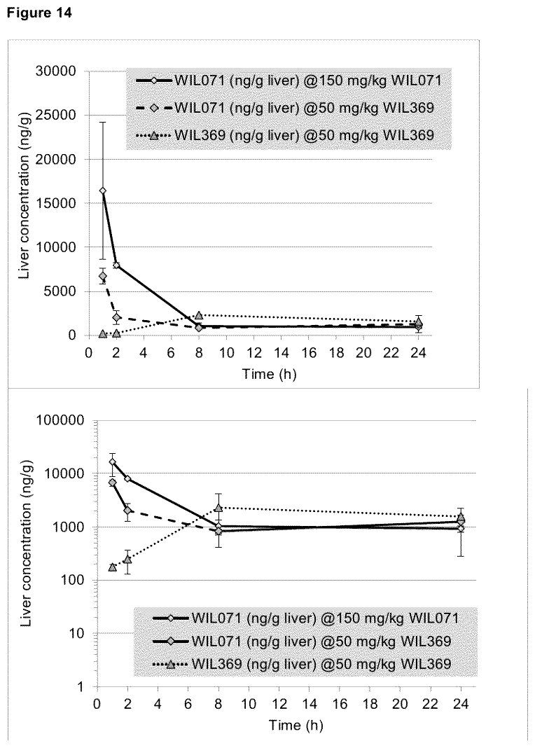

FIG. 14 shows that a quick degradation takes place in the liver; it is the only place where WIL-369 is found.

### REFERENCES

### Apoptosis

G. M. Cohen (1997), Caspases: the executioners of apoptosis, Biochem. J. 326, 1-16.
F. Eβmann, T. Wieder, A. Otto, E.-C. Müller, B. Dörken, P. T. Daniel (2000), The GDP dissociation inhibitor, D4-GDI (Rho-GDI 2), but not the homologous Rho-GDI 1, is cleaved by caspase-3 during drug-induced apoptosis, Biochem. J. 346, 777-783.
Y. A. Hannun (1997), Apoptosis and the dilemma of cancer therapy, Blood 89, 1845-1853.
J. A. Hickman (1996), Apoptosis and chemotherapy resistance, Eur. J. Cancer 32A, 921-926.
M. Raisova, M. Bektas, T. Wieder, P. T. Daniel, J. Eberle, C. E. Orfanos, C. C. Geilen (2000), Resistance to CD95/Fas-induced and ceramide-mediated apoptosis of human melanoma cells is caused by a defective mitochondrial cytochrome c release, FEBS Lett. 473, 27-32.
   Physiological Function of glucocorticoids:
Munck A, Guyre P M, Holbrook N J (1984). Physiological functions of glucocorticoids in stress and their relation to pharmacological actions. Endocr Rev, 5, 25-44.

### Glucocorticoids for the Therapy of Malignant Diseases:

Keith B D (2008). Systematic review of the clinical effect of glucocorticoids on nonhematologic malignancy. BMC Cancer, 8, 84.
   Prednisone Good Vs. Poor Responder:
Dördelmann M, Reiter A, Borkhardt A, Ludwig W D, Götz N, Viehmann S, Gadner H, Riehm H, Schrappe M (1999). Prednisone response is the strongest predictor of treatment outcome in infant acute lymphoblastic leukemia. Blood, 94, 1209-1217.
Pufall M A (2015). Glucocorticoids and Cancer. Adv Exp Med Biol, 872, 315-333.

### Mechanism of Action:

### In General:

Geley S, Fiegl M, Hartmann B L, Kofler R (1996). Genes mediating glucocorticoid effects and mechanisms of their regulation. Reviews of physiology, biochemistry and pharmacology, 128, 1-97.
Giguére V, Hollenberg S M, Rosenfeld M G, Evans R M (1986). Functional domains of the human glucocorticoid receptor. Cell, 46, 645-652.
Kuida K, Haydar T F, Kuan C—Y, Gu Y, Taya C, Karasuyama H, Su M S-S, Rakic P, Flavell P A (1998). Reduced Apoptosis and Cytochrome c-Mediated Caspase Activation in Mice Lacking Caspase 9. Cell, 94, 325-337.
Mattern J, Büchler MW, Herr I (2007). Cell cycle arrest by glucocorticoids may protect normal tissue and solid tumors from cancer therapy. Cancer Biol Ther, 6, 1345-1354.
Greenstein S, Ghias K, Krett N L, Rosen S T (2002). Mechanisms of glucocorticoid-mediated apoptosis in hematological malignancies. Clin Cancer Res, 8, 1681-1694.
Miyashita T, Nagao K, Krajewski S, Salvesen G S, Reed J C, Inoue T, Yamada M (1998). Investigation of glucocorticoid-induced apoptotic pathway: processing of caspase-6 but not caspase-3. Cell death and differentiation, 5, 1034-1041.
Planey S L, Abrams M T, Robertson N M, Litwack G (2003). Role of apical caspases and glucocorticoid-regulated genes in glucocorticoid-induced apoptosis of pre-B leukemic cells. Cancer Res, 63, 172-178.
Rathmell J C, Lindsten T, Zong W-X, Cinalli R M, Thompson C B (2002). Deficiency in Bak and Bax perturbs thymic selection and lymphoid homeostasis. Nat Immunol, 3, 932-939.
Almond J B, Snowden R T, Hunter A, Dinsdale D, Cain K, Cohen G M (2001). Proteasome inhibitor-induced apoptosis of B-chronic lymphocytic leukaemia cells involves cytochrome c release and caspase activation, accompanied by formation of an approximately 700 kDa Apaf-1 containing apoptosome complex. Leukemia, 15, 1388-1397.
   Interactions with Transcription Factors:
Herrlich P (2001). Cross-talk between glucocorticoid receptor and AP-1. Oncogene, 20, 2465-2475.

### Cell Cycle Arrest:

Mattern J, Büchler MW, Herr I (2007). Cell cycle arrest by glucocorticoids may protect normal tissue and solid tumors from cancer therapy. Cancer Biol Ther, 6, 1345-1354.
Greenstein S, Ghias K, Krett N L, Rosen S T (2002). Mechanisms of glucocorticoid-mediated apoptosis in hematological malignancies. Clin Cancer Res, 8, 1681-1694.
Rogatsky I, Trowbridge J M, Garabedian M J (1997). Glucocorticoid receptor-mediated cell cycle arrest is achieved through distinct cell-specific transcriptional regulatory mechanisms. Mol Cell Biol, 17, 3181-3193.

### ER Stress, ROS, Autophagy:

Aoki S, Morita M, Hirao T, Yamaguchi M, Shiratori R, Kikuya M, Chibana H, Ito K (2017). Shift in energy metabolism caused by glucocorticoids enhances the effect of cytotoxic anti-cancer drugs against acute lymphoblastic leukemia cells. Oncotarget, 8, 94271-94285.
Liu W, Zhao Z, Na Y, Meng C, Wang J, Bai R (2018). Dexamethasone-induced production of reactive oxygen species promotes apoptosis via endoplasmic reticulum stress and autophagy in MC3T3-E1 cells. Int J Mol Med, 41, 2028-2036.
Yang J, Wu Q, Lv J, Nie H (2017). 4-Phenyl butyric acid prevents glucocorticoid-induced osteoblast apoptosis by attenuating endoplasmic reticulum stress. Journal of bone and mineral metabolism, 35, 366-374.

1. Medicaments based on 7,19-epoxy-steroids comprising a compound of general formula I, 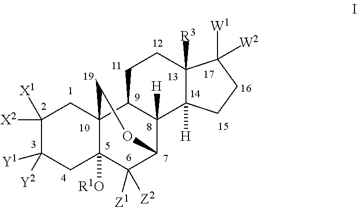
or a physiologically tolerable salt thereof, wherein in the formula:
R¹=H; methyl, aryl-CH₂—, R^{x}—C(═O)—, or R^{x}—O—C(═O)—; or R¹O is omitted if a double bond exists between 4-5;
W¹, W² are independently H, OH, CHR²OH, CHR²R^{x}, CR²R^{x}OH, or W¹ and W² are together O,
R²=saturated or unsaturated, optionally branched, alkyl radicals with 4 to 9 carbon atoms, wherein such alkyl radicals may also have a cyclopropane or cyclobutane substructure;
R³=methyl or ethyl,
X¹, X² are independently R^{x} or OR⁵, or together form O, NOR⁴;
Y¹, Y² are independently R^{x} or OR⁵, or together form O, NOR⁴;
or X¹ and Y¹ together form a double bond or epoxy group;
Z¹, Z² are independently R^{x} or OR⁵, or together form O, NOR⁴, CH₂;
R⁴ are independently H, alkyl or aryl; and
R⁵ are independently H; alkyl, aryl-CH₂—, R^{x}—C(═O)— or R^{x}—O—C(═O)—, SO₃H, SO₃CH₃, PO(OH)₂ or glycosyl;
wherein each R^{x} is independently H, saturated or unsaturated, optionally branched, alkyl, saturated or unsaturated, optionally branched, substituted alkyl, aryl, heteroaryl, PEG, SO₃H, PO(OH)₂, or glycosyl;
and wherein up to 3 hydrogens may be substituted by fluorine.

2. The medicament of claim 1, wherein formula I has formula A, 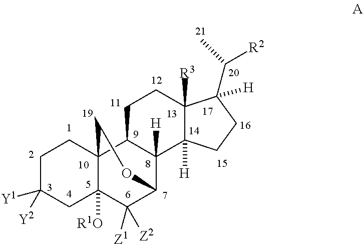
and the substituents are as defined in claim 1.

3. The medicament according to claim 1, wherein
(i) R^{x} has from 1 to 10 carbon atoms, and/or
(ii) said substituted alkyl in R^{x} has —OR′ or C(═O)OR′ as a substituent, and R′ is H or alkyl or a protective group, or
(iii) R¹ is H, Z¹, Z² are O, R³ is methyl.

4. The medicament according to claim 1, wherein
Y¹ is H, Y² is OH, or Y¹ is H, Y² is OR⁵, wherein R⁵ is R^{x}—C(═O)— or R^{x}—O—C(═O)—, and R^{x} is H, alkyl, substituted alkyl, aryl, or heteroaryl.

5. The medicament according to claim 1, wherein R^{x} is H, alkyl, and R² is 4-methylpentyl.

6. The medicament according to claim 1, wherein R¹ is H; methyl.

7. The medicament according to claim 1,
(i) wherein R⁴ has up to 10 carbon atoms,
(ii) wherein R⁵ has up to 10 carbon atoms or
(iii) wherein R⁴ has up to 10 carbon atoms and R⁵ has up to 10 carbon atoms.

8. The medicament according to claim 1, wherein R^{x} has up to 10 carbon atoms.

9. The medicament according to claim 1, wherein R^{x} is alkyl or substituted alkyl.

10. A process for preparing the compound of formula I of claim 1 comprising step a, followed by conversion of functional groups: 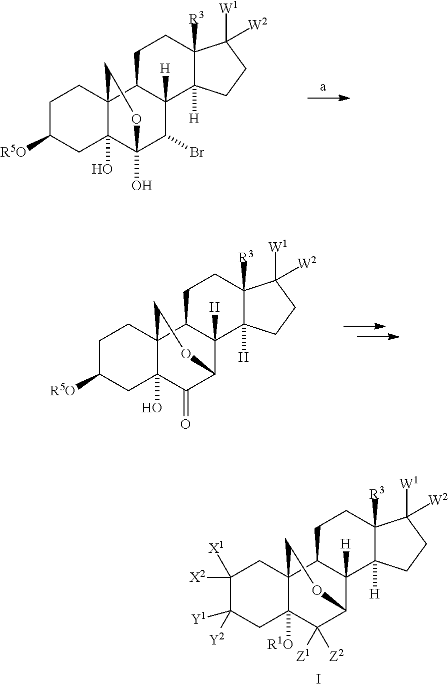
wherein step a is performed under basic conditions.

11. A method of treating malignant diseases, benign or semimalignant skin diseases comprising the step of administering the compound of formula I of claim 1 to a patient in need thereof.

12. The method of claim 11, wherein said malignant diseases affect bone marrow or other hematopoietic organs, or are solid tumors or epithelial tumors.

13. The method of claim 11, wherein said benign or semimalignant skin diseases are psoriasis vulgaris, keloids, or basaliomas.

14. A method of treating inflammatory and chronic inflammatory diseases comprising the step of administering the compound of formula I of claim 1 to a patient in need thereof.

15. The method according to claim 11, wherein said treating is combined with a cytostatic.

16. The method of claim 12 wherein said solid tumors are brain tumors of grade 1-grade 4.

17. The method of claim 16 wherein said solid tumors are glioblastoma or medulloblastoma.

18. A method of treating a disease comprising the step of administering the compound of formula I of claim 1 to a patient in need thereof in immunosuppressive therapy.

19. A method of treating a disease comprising the step of administering the compound of formula I of claim 1 to a patient in need thereof in antiviral, antibacterial, antimycotic, antiprotozoan, or antihelminthic therapy.

20. The method of claim 15 wherein said cytostatic is selected from the group consisting of cytarabin and vincristine.
